(19) United States
(12) Patent Application Publication
(10) Pub. No.: US 2023/0365646 A1
(43) Pub. Date: Nov. 16, 2023

(54) AZIDE INSULIN ANALOGUES

(71) Applicant: Merck Sharp & Dohme LLC, Rahway, NJ (US)

(72) Inventors: Sobhana Babu Boga, South Plainfield, NJ (US); Ahmet Kekec, Hoboken, NJ (US); Songnian Lin, Holmdel, NJ (US); Craig A. Parish, Tenafly, NJ (US); Weijuan Tang, Scotch Plains, NJ (US); Lin Yan, East Brunswick, NJ (US)

(73) Assignee: Merck Sharp & Dohme LLC, Rahway, NJ (US)

(21) Appl. No.: 18/362,417

(22) Filed: Jul. 31, 2023

Related U.S. Application Data
(63) Continuation of application No. 17/045,197, filed on Oct. 5, 2020.

(51) Int. Cl. C07K 14/62; C07K 1/00

(52) U.S. Cl. CPC C07K 14/62; C07K 1/006

(57) ABSTRACT

The present invention relates to insulin analogues and processes of making such insulin analogues by direct conversion of a free amine to an azide via diazo-transfer with an azotransfer agent.

### REFERENCE TO SEQUENCE LISTING SUBMITTED ELECTRONICALLY

The sequence listing of the present application is submitted electronically via EFS-Web as an ASCII formatted sequence listing with a file name “24573WOPCT-SEQLIST-11MAR2019”, creation date of Mar. 11, 2019, and a size of 4.82 KB. This sequence listing submitted via EFS-Web is part of the specification and is herein incorporated by reference in its entirety.

### FIELD

The present invention relates to azide insulin analogues and processes of making such azide insulin analogues by direct conversion of a free amine to an azide via diazo-transfer with an azotransfer agent. The present invention further relates to triazole conjugates of insulin and processes of making such triazole conjugates of insulin by reacting an azide insulin analog with an alkyne.

### BACKGROUND

Late-Stage Functionalization (LSF) of peptides and proteins is directed at the selective modification of amino acids and has the potential to impact medicinal chemistry, chemical biology and protein structural studies. Azidation of peptides and proteins such as insulin or insulin analogues provides key intermediates that possesse a moiety with distinct bio-orthogonal reactivity. These azide insulin analog intermediates then can be utilized for a wide range of bio-conjugation reactions in chemical biology that include azide-alkyne Click Chemistry, Staudinger ligation, photochemistry, etc. Specifically, direct azidation allows further development of tools to effect PK (such as pegylation or glycosylation), imaging (such as biotinylation) or to allow for further chemistries at a previously inaccessible site on the peptide (such as preparing insulin dimers).

### BRIEF SUMMARY OF THE INVENTION

The present invention provides azide insulin analogues and processes for making such analogues by direct conversion of an amine to an azide via diazo-transfer using an azotransfer agent.

Thus, the present invention provides the following embodiments, a process of making an azide insulin analog comprising directly converting a free amine on insulin or insulin analog to an azide via diazo-transfer using imidazole-1-sulfonyl azide. In certain embodiments of the processes described herein, the insulin or insulin analog is recombinant human insulin. In certain embodiments, wherein the processes described herein use recombinant human insulin, the free amine on the B29- lysine on human insulin is converted to an azide. In certain embodiments, wherein the processes described herein use recombinant human insulin, the free amine A1-glycine on human insulin is converted to an azide. In certain embodiments, wherein the processes described herein use recombinant human insulin, the free amine B1-phenyl alanine on human insulin is converted to an azide. In certain embodiments, wherein the processes described herein use recombinant human insulin, all three of the free amines on human insulin, B29-lysine A1-glycine and B1-phenyl alanine are converted to azides. In certain embodiments, wherein the processes described herein use recombinant human insulin, two of the three of the free amines on the human insulin, B29-lysine A1-glycine or B1-phenyl alanine, are converted to azides.

In certain embodiments, the present invention also provides a process of making an azide insulin analog comprising: introducing insulin or an insulin analog to a solvent system having a pH between 5-10; adding an azotransfer agent; and maintaining a pH between 5-10. In certain embodiments of the processes described herein, the azotransfer agent is 2-azido-1, 3-dimethylimidazolinium PF6, imidazole-1-sulfonyl azide BF4, 1H-imidazole-1-sulfonyl azide HCl and 1H-imidazole-1-sulfonyl azide sulfate. In certain embodiment of the processes described herein, the solvent system is a mixture of water and methanol. In yet other embodiments of the processes described herein, sodium bicarbonate is further added to the solvent system. In still other embodiment of the processes described herein, copper(II) sulfate.5H₂0 is further added to the solvent system. In still other embodiments of the processes described herein, the pH is maintained between 8-9.

Also described herein are triazole conjugates of insulin and processes of making such triazole conjugates of insulin by reacting an azide insulin analog with an alkyne. In certain embodiments, the azide insulin analog is an azide insulin analog described herein.

### FIGURES

FIG. 1 shows four peptide digests (labelled as F4, F1-F5, F2-F6 and F3) that were generated after Glu-C digestion.

### DESCRIPTION

### Definitions

Insulin - as used herein, the term means the active principle of the pancreas that affects the metabolism of carbohydrates in the animal body and which is of value in the treatment of diabetes mellitus. The term includes synthetic and biotechnologically derived products that are the same as, naturally occurring insulins in structure, use, and intended effect and are of value in the treatment of diabetes mellitus. The term is a generic term that designates the 51 amino acid heterodimer comprising the A-chain peptide having the amino acid sequence shown in SEQ ID NO: 1 and the B-chain peptide having the amino acid sequence shown in SEQ ID NO: 2, wherein the cysteine residues a positions 6 and 11 of the A chain are linked in a disulfide bond, the cysteine residues at position 7 of the A chain and position 7 of the B chain are linked in a disulfide bond, and the cysteine residues at position 20 of the A chain and 19 of the B chain are linked in a disulfide bond.

Insulin analog or analogue - the term as used herein includes any heterodimer analogue or single-chain analogue that comprises one or more modification(s) of the native A-chain peptide and/or B-chain peptide of insulin. Modifications include but are not limited to substituting an amino acid for the native amino acid at a position selected from A4, A5, A8, A9, A10, A12, A13, A14, A15, A16, A17, A18, A19, A21, B1, B2, B3, B4, B5, B9, B10, B13, B14, B15, B16, B17, B18, B20, B21, B22, B23, B26, B27, B28, B29, and B30; deleting any or all of positions B1-4 and B26-30; or conjugating directly or by a polymeric or non-polymeric linker one or more acyl, polyethylglycine (PEG), or saccharide moiety (moieties); or any combination thereof. Examples of insulin analogues include but are not limited to the heterodimer and single-chain analogues disclosed in published international applications WO2010/0080606, WO2009/099763, and WO2010/080609, the disclosures of which are incorporated herein by reference. Examples of single-chain insulin analogues also include but are not limited to those disclosed in published International Applications WO96/34882, WO95/516708, WO2005/054291, WO2006/097521, WO2007/104734, WO2007/104736, WO2007/104737, WO2007/104738, WO2007/096332, WO2009/132129; U.S. Pat. Nos. 5,304,473 and 6,630,348; and Kristensen et al., Biochem. J. 305: 981-986 (1995), the disclosures of which are each incorporated herein by reference.

The term further includes single-chain and heterodimer polypeptide molecules that have little or no detectable activity at the insulin receptor but which have been modified to include one or more amino acid modifications or substitutions to have an activity at the insulin receptor that has at least 1%, 10%, 50%, 75%, or 90% of the activity at the insulin receptor as compared to native insulin and which further includes at least one N-linked glycosylation site. In particular aspects, the insulin analogue is a partial agonist that has less than 80% (or 70%) activity at the insulin receptor as does native insulin. These insulin analogues, which have reduced activity at the insulin growth hormone receptor and enhanced activity at the insulin receptor, include both heterodimers and single-chain analogues.

Single-chain insulin or single-chain insulin analog - as used herein, the term encompasses a group of structurally-related proteins wherein the A-chain peptide or functional analogue and the B-chain peptide or functional analogue are covalently linked by a peptide or polypeptide of 2 to 35 amino acids or non-peptide polymeric or non-polymeric linker and which has at least 1%, 10%, 50%, 75%, or 90% of the activity of insulin at the insulin receptor as compared to native insulin. The single-chain insulin or insulin analogue further includes three disulfide bonds: the first disulfide bond is between the cysteine residues at positions 6 and 11 of the A-chain or functional analogue thereof, the second disulfide bond is between the cysteine residues at position 7 of the A-chain or functional analogue thereof and position 7 of the B-chain or functional analogue thereof, and the third disulfide bond is between the cysteine residues at position 20 of the A-chain or functional analogue thereof and position 19 of the B-chain or functional analogue thereof.

Azide insulin analog - as used herein, the term encompasses insulin analogues that were made according to the processes described herein.

Triazole conjugate of insulin - as used herein, the term encompasses insulin or insulin analogues that are conjugated to a triazole by reacting an azide insulin analog with an alkyne.

Pharmaceutically acceptable carrier - as used herein, the term includes any of the standard pharmaceutical carriers, such as a phosphate buffered saline solution, water, emulsions such as an oil/water or water/oil emulsion, and various types of wetting agents suitable for administration to or by an individual in need. The term also encompasses any of the agents approved by a regulatory agency of the US Federal government or listed in the US Pharmacopeia for use in animals, including humans.

Pharmaceutically acceptable salt - as used herein, the term refers to salts of compounds that retain the biological activity of the parent compound, and which are not biologically or otherwise undesirable. Many of the compounds disclosed herein are capable of forming acid and/or base salts by virtue of the presence of amino and/or carboxyl groups or groups similar thereto.

Pharmaceutically acceptable base addition salts can be prepared from inorganic and organic bases. Salts derived from inorganic bases, include by way of example only, sodium, potassium, lithium, ammonium, calcium, zinc and magnesium salts. Salts derived from organic bases include, but are not limited to, salts of primary, secondary and tertiary amines.

Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like.

Effective or therapeutically effective amount - as used herein refers to a nontoxic but sufficient amount of an insulin analog to provide the desired effect. For example one desired effect would be the prevention or treatment of hyperglycemia. The amount that is “effective” will vary from subject to subject, depending on the age and general condition of the individual, mode of administration, and the like. Thus, it is not always possible to specify an exact “effective amount.” It is not always possible to determine the optimal effective amount prior to administration to or by an individual in need thereof. However, an appropriate “effective” amount in any individual case may be determined by one of ordinary skill in the art using routine experimentation.

Parenteral - as used herein, the term means not through the alimentary canal but by some other route such as intranasal, inhalation, subcutaneous, intramuscular, intraspinal, or intravenous.

### Azidation Process

The present invention is directed to azide insulin analogues and processes of making such azide insulin analogues. The azidation processes described herein include direct conversion of a free amine of insulin or an insulin analog to an azide via diazo-transfer using an azotransfer agent. The process is an improvement over prior processes described in U.S. Pat. Application 15/061029, since the azidation is a single-step process.

In certain embodiments the process described herein includes the following steps:
introducing insulin or an insulin analog to a solvent system having a pH between 5-10;
adding an azotransfer agent to the insulin or insulin analog solvent system; and
maintaining the environment at a pH between 5-10.

In the processes described herein, the azotransfer agent can be selected from imidazole-1-sulfonyl azide. HCl, BF₄, HSO₄, (2-azido-1-methyl-1H-imidazol-3-ium-3-yl)methanide compound with hexafluoro-16-phosphane, represented by the following formulas:

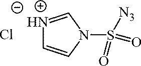 [SMILES: Cl ; [N-]=[N+]=NS(=O)(=O)n1ccnc1]

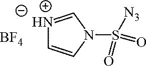 [SMILES: "BF4" ; [N-]=[N+]=NS(=O)(=O)n1ccnc1]

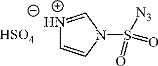 [SMILES: "HSO4" ; [N-]=[N+]=NS(=O)(=O)n1ccnc1]

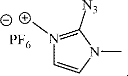

In the process described herein, the solvent system can comprise of any suitable solvent including, but not limited to, water, methanol (MeOH), isopropyl alcohol (iPrOH), tert- butanol (tBuOH), tert-amyl alcohol (tAmylOH) and dimethylacetamide (DMAc), or a combination thereof.

In certain embodiment, the solvent system of the processes described herein can be a combination of two or more solvents in optimal rations. Suitable ratios include, but are not limited to, are 9:1, 4:1, 7:3, 3:2, 1:1, 2:3, 3:7; 1:4 and 1:9.

In certain embodiments, the solvent system is a combination of methanol and water. A suitable ratio of water and methanol can include, but is not limited to, are 9:1, 4:1, 7:3, 3:2, 1:1, 2:3, 3:7; 1:4 and 1:9. In certain embodiments, the solvent system is a combination of water and methanol in a ratio of 9:1.

In certain embodiments, the solvent system is methanol. In certain embodiments, the solvent system is dimethylacetamide. In certain embodiments, the solvent system is isopropyl alcohol.

In the processes described herein the solvent system is at a pH between 5-10. In certain embodiments the solvent system used in the processes described herein is at a pH between 6-10. In certain embodiments the solvent system used in the processes described herein is at a pH between 7-10. In certain embodiments the solvent system used in the processes described herein is at a pH between 8-10. In certain embodiments the solvent system used in the processes described herein is at a pH between 9-10. In certain embodiments the solvent system used in the processes described herein is at a pH between 6-9. In certain embodiments the solvent system used in the processes described herein is at a pH between 6-8. In certain embodiments the solvent system used in the processes described herein is at a pH between 6-7. In certain embodiments the solvent system used in the processes described herein is at a pH between 8-9.

In certain embodiments the solvent system used in the processes described herein is at a pH of 6. In certain embodiments the solvent system used in the processes described herein is at a pH of 7. In certain embodiments the solvent system used in the processes described herein is at a pH of 8. In certain embodiments the solvent system used in the processes described herein is at a pH of 9. In certain embodiments the solvent system used in the processes described herein is at a pH of 10.

The solvent system can be adjusted or kept at the desired pH by using pH buffers. Buffers can be added at the beginning of the reaction or during the reaction, as needed, to keep the pH of the system at the desired pH. Suitable buffers include, but are not limited to, sodium bicarbonate, sodium potassium phosphate, sodium acetate, disodium phosphate, (NaHPO₄/Na₂H₂PO₄) or sodium bicarbonate/sodium carbonate (Na₂CO₃/NaHCO₃).

In certain embodiments of the processes described herein, the buffered solvent system can also include copper (II) sulfate.5H₂0. In certain embodiments of the processes described herein, the buffered solvent system does not include copper (II) sulfate.5H₂0.

Though the chemistry processes described herein can be used on any peptide or protein with a free amine, the processes and examples described herein are used in conjunction with insulin or insulin analogues.

Suitable types of insulin that can be used in the processes described herein include, but are not limited to, recombinant human insulin (RHI) and independently native human insulin.

As for insulin analogues, one type of insulin analog that can be used in the processes described herein, “monomeric insulin analog,” is well known in the art. These are fast-acting analogues of human insulin, including, for example, insulin analogues wherein:
(a) the amino acyl residue at position B28 is substituted with Asp, Lys, Leu, Val, or Ala, and the amino acyl residue at position B29 is Lys or Pro;
(b) the amino acyl residues at any of positions B27 and B30 are deleted or substituted with a nonnative amino acid.

In one embodiment an insulin analog that can be used in the processes described herein, is provided comprising an Asp substituted at position B28 (e.g., insulin aspart (NOVOLOG); see SEQ ID NO:3) or a Lys substituted at position 28 and a proline substituted at position B29 (e.g., insulin lispro (HUMALOG); see SEQ ID NO:4). Additional monomeric insulin analogues are disclosed in Chance, et al., U.S. Pat. No. 5,514,646; Chance, et al., U.S. Pat. application Ser. No. 08/255,297; Brems, et al., Protein Engineering, 5:527-533 (1992); Brange, et al., EPO Publication No. 214,826 (published Mar. 18, 1987); and Brange, et al., Current Opinion in Structural Biology, 1:934-940 (1991). These disclosures are expressly incorporated herein by reference for describing monomeric insulin analogues.

Insulin analogues that can be used in the processes described herein may also have replacements of the amidated amino acids with acidic forms. For example, Asn may be replaced with Asp or Glu. Likewise, Gln may be replaced with Asp or Glu. In particular, Asn(A18), Asn(A21), or Asp(B3), or any combination of those residues, may be replaced by Asp or Glu. Also, Gln(A15) or Gln(B4), or both, may be replaced by either Asp or Glu.

In one embodiment the insulin analogues that can be used in the processes described herein have the A chain comprising amino acid sequence GIVEQCCTSICSLYQLENYCN (SEQ ID NO: 1 and the B chain comprising amino acid sequence FVNQHLCGSHLVEALYLV CGERGFFYTPKT (SEQ ID NO: 2) or a carboxy shortened sequence thereof having B30 deleted, and analogues of those sequences wherein each sequence is modified to comprise one to five amino acid substitutions at positions corresponding to native insulin positions selected from A5, A8, A9, A10, A14, A15, A17, A18, A21, B1, B2, B3, B4, B5, B9, B10, B13, B14, B20, B22, B23, B26, B27, B28, B29 and B30, with the proviso that at least one of B28 or B29 is lysine. In one embodiment the amino acid substitutions of the insulin analogues that can be used in the processes described herein are conservative amino acid substitutions. Suitable amino acid substitutions at these positions that do not adversely impact insulin’s desired activities are known to those skilled in the art, as demonstrated, for example, in Mayer, et al., Insulin Structure and Function, Biopolymers. 2007;88(5):687-713, the disclosure of which is incorporated herein by reference.

In accordance with one embodiment, the insulin analog peptides that can be used in the processes described herein may comprise an insulin A chain and an insulin B chain or analogues thereof, wherein the A chain comprises an amino acid sequence that shares at least 70% sequence identity (e.g., 70%, 75%, 80%, 85%, 90%, 95%) over the length of the native peptide, with GIVEQCCTSICSLYQLENYCN (SEQ ID NO: 1) and the B chain comprises an amino acid sequence that shares at least 60% sequence identity (e.g., 60%, 65%, 70%, 75%, 80%, 85%, 90%, 95%) over the length of the native peptide, with FVNQHLCGSHLVEALYLVCGERGFFYTPKT (SEQ ID NO: 2) or a carboxy shortened sequence thereof having B30 deleted.

Additional amino acid sequences can be added to the amino terminus of the B chain or to the carboxy terminus of the A chain of the insulin polypeptides of insulin analogues that can be used in the processes described herein. For example, a series of negatively charged amino acids can be added to the amino terminus of the B chain, including for example a peptide of 1 to 12, 1 to 10, 1 to 8 or 1 to 6 amino acids in length and comprising one or more negatively charged amino acids including for example glutamic acid and aspartic acid. In one embodiment the B chain amino terminal extension comprises 1 to 6 charged amino acids. In accordance with one embodiment the insulin polypeptides disclosed comprise a C-terminal amide or ester in place of a C-terminal carboxylate on the A chain.

In various embodiments, the insulin analogues that can be used in the processes described herein have an isoelectric point that has been shifted relative to human insulin. In some embodiments, the shift in isoelectric point is achieved by adding one or more arginine, lysine, or histidine residues to the N-terminus of the insulin A-chain peptide and/or the C-terminus of the insulin B-chain peptide. Examples of such insulin polypeptides include Arg^{A0}-human insulin, Arg^{B31}Arg^{B32}-human insulin, Gly^{A21}Arg^{B31}Arg^{B32}-human insulin, Arg^{A0}Arg^{B31}Arg^{B32}-human insulin, and Arg^{A0}Gly^{A21}Arg^{B31}Arg^{B32}-human insulin. By way of further example, insulin glargine (LANTUS; see SEQ ID NOs: 5 and 6) is an exemplary long-acting insulin analog in which Asn^{A21} has been replaced by glycine, and two arginine residues have been covalently linked to the C-terminus of the B-peptide. The effect of these amino acid changes was to shift the isoelectric point of the molecule, thereby producing a molecule that is soluble at acidic pH (e.g., pH 4 to 6.5) but insoluble at physiological pH. When a solution of insulin glargine is injected into the muscle, the pH of the solution is neutralized and the insulin glargine forms microprecipitates that slowly release the insulin glargine over the 24 hour period following injection with no pronounced insulin peak and thus a reduced risk of inducing hypoglycemia. This profile allows a once-daily dosing to provide a patient’s basal insulin. Thus, in some embodiments, the insulin analogues that can be used in the processes described herein comprise an A-chain peptide wherein the amino acid at position A21 is glycine and a B-chain peptide wherein the amino acids at position B31 and B32 are arginine. The present disclosure encompasses all single and multiple combinations of these mutations and any other mutations that are described herein (e.g., Gly^{A21}-human insulin, Gly^{A21}Arg^{B31}-human insulin, Arg^{B31}Arg^{B32}-human insulin, Arg^{B31}-human insulin).

In certain embodiments of the insulin analogues that can be used in the processes described herein, one or more amidated amino acids of the insulin analog are replaced with an acidic amino acid, or another amino acid. For example, asparagine may be replaced with aspartic acid or glutamic acid, or another residue. Likewise, glutamine may be replaced with aspartic acid or glutamic acid, or another residue. In particular, Asn^{A18}, Asn^{A21}, or Asn^{B3}, or any combination of those residues, may be replaced by aspartic acid or glutamic acid, or another residue. Gln^{A15} or Gln^{B4}, or both, may be replaced by aspartic acid or glutamic acid, or another residue. In particular aspects of the insulin receptor partial agonists, the insulin analogues have an aspartic acid, or another residue, at position A21 or aspartic acid, or another residue, at position B3, or both.

One skilled in the art will recognize that it is possible to replace yet other amino acids in the insulin analog that can be used in the processes described herein with other amino acids while retaining biological activity of the molecule. For example, without limitation, the following modifications are also widely accepted in the art: replacement of the histidine residue of position B10 with aspartic acid (His^{B10} to Asp^{B10}); replacement of the phenyl alanine residue at position B1 with aspartic acid (Phe^{B1} to Asp^{B1}); replacement of the threonine residue at position B30 with alanine (ThrB30 toAlaB30); replacement of the tyrosine residue at position B26 with alanine (Tyr^{B26} to Ala^{B26}); and replacement of the serine residue at position B9 with aspartic acid (Ser^{B9} to Asp^{B9}).

In certain embodiments, the insulin analogues that can be used in the processes described herein may be acylated with a fatty acid. That is, an amide bond is formed between an amino group on the insulin analog and the carboxylic acid group of the fatty acid. The amino group may be the alpha-amino group of an N-terminal amino acid of the insulin analog, or may be the epsilon-amino group of a lysine residue of the insulin analog. The insulin analog may be acylated at one or more of the three amino groups that are present in wild-type human insulin may be acylated on lysine residue that has been introduced into the wild-type human insulin sequence. In certain embodiments, the insulin analogues that can be used in the processes described herein may be acylated at position A1, B1, or both A1 and B1. In certain embodiments, the fatty acid is selected from myristic acid (C₁₄), pentadecylic acid (C₁₅), palmitic acid (C₁₆), heptadecylic acid (C₁₇) and stearic acid (C₁₈).

Examples of insulin analogues that can be used in the processes described herein can be found for example in published International Application WO9634882, WO95516708; WO2010/0080606, WO2009/099763, and WO2010/080609, US Pat. No. 6,630,348, and Kristensen et al., Biochem. J. 305: 981-986 (1995), the disclosures of which are incorporated herein by reference). In further embodiments, the in vitro glycosylated or in vivo N-glycosylated insulin analogues may be acylated and/or pegylated.

In certain embodiments, an insulin analog that can be used in the processes described herein is provided wherein the A chain of the insulin peptide comprises the sequence GIVEQCCX₈SICSLYQLX₁₇NX₁₉CX₂₃ (SEQ ID NO: 7) and the B chain comprises the sequence X₂₅LCGX₂₉X₃₀LVEALYLVCGERGFFYTX₃₁X₃₂ (SEQ ID NO: 8) wherein
X₈ is threonine or histidine;
X₁₇ is glutamic acid or glutamine;
X₁₉ is tyrosine, 4-methoxy-phenyl alanine, or 4-amino phenyl alanine;
X₂₃ is asparagine or glycine;
X₂₅ is histidine or threonine;
X₂₉ is alanine, glycine or serine;
X₃₀ is histidine, aspartic acid, glutamic acid, homocysteic acid, or cysteic acid;
X₃₁ is proline or lysine; and
X₃₂ is proline or lysine, with the proviso that at least one of X₃₁ or X₃₂ is lysine.

In a further embodiment, an insulin analog that can be used in the processes described herein the B chain comprises the sequence X₂₂VNQX₂₅LCGX₂₉X₃₀LVEALYLVCGERGFFYT-X₃₁X₃₂X₃₃X₃₄X₃₅ (SEQ ID NO: 9) wherein
X₂₂ is or phenyl alanine and desamino-phenyl alanine;
X₂₅ is histidine or threonine;
X₂₉ is alanine, glycine, or serine;
X₃₀ is histidine, aspartic acid, glutamic acid, homocysteic acid, or cysteic acid;
X₃₁ is aspartic acid, proline, or lysine;
X₃₂ is lysine or proline;
X₃₃ is threonine, alanine, or absent;
X₃₄ is arginine or absent; and
X₃₅ is arginine or absent;
With the proviso at least one of X₃₁ or X₃₂ is lysine.

In certain embodiments, the processes described herein include: combining insulin or an insulin analog with sodium bicarbonate and copper (II) sulfate.5H₂0 dissolved in water and methanol;
adding 1H-imidazole-1-sulfonyl azide hydrochloride; and
maintaining the solution as pH 8-9 by adding aqueous saturated NaHCO₃ solution.

In certain embodiments, one or all of the existing free amines on the insulin or insulin analog can be converted to an azide. Free amines on the insulin or insulin analog that will not be converted to an azide can first be protected. Various protection methods are known in the art.

Suitable amino-protecting groups include methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, phenothiazinyl-(10)-carbonyl derivative, N′-p-toluenesulfonylaminocarbonyl derivative, N′-phenylaminothiocarbonyl derivative, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxycarbonylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, 2,4,6-trimethylbenzyl carbamate, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxycarbonylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, o-(benzoyloxymethyl)benzamide, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N-(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentacarbonylchromium- or tungsten)carbonyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, 3-nitropyridinesulfenamide (Npys), p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Optimization of the reaction conditions (reagents/pH/solvents/± CuSO₄.5H₂O) can provide site-selective azidation of insulin at each amine position.

### Processes of Making Azide Insulin Analogues With Recombinant Human Insulin (RHI)

In certain embodiments, the present invention relates to azide insulin analogues and processes of making such azide insulin analogues by direct conversion of a free amine of RHI to an azide via diazo-transfer using imidazole-1-sulfonyl azide.

RHI has three different amino acid (AA) free amines and each have different pKa values, A1-glycine (α—NH2, Pka 8.4 on the A-chain and B29-lysine (ε—NH2, Pka 11.1), B1-phenyl alanine (α—NH2, Pka 7.1) on the B-chain.

In certain embodiments described herein, one, two or three of the free amines on RHI is converted to mono azide, di azide and tri-azido insulin. In certain embodiments described herein, one of the free amines on RHI is converted to a mono azide insulin. In certain embodiments described herein, two of the free amines on RHI are converted to di azide insulin. In certain embodiments described herein, all three of the free amines on RHI are converted to tri-azido insulin.

In certain embodiments described herein, the free amine B29-lysine on RHI is converted to an azide. In other embodiments, the glycine-A1 termini amine is converted to an azide. In still another embodiment, the phenyl alanine-B1 termini amine is converted to an azide.

In embodiments wherein less than three of the free amines in RHI are converted to azides using the processes described herein, the remaining free amines can be protected prior to undergoing the chemistry described herein.

Free amines on the recombinant human insulin that will not be converted to an azide can first be protected. Various protection methods are known in the art.

Suitable amino-protecting groups include methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, phenothiazinyl-(10)-carbonyl derivative, N′-p-toluenesulfonylaminocarbonyl derivative, N′-phenylaminothiocarbonyl derivative, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxycarbonylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, 2,4,6-trimethylbenzyl carbamate, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxycarbonylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, o-(benzoyloxymethyl)benzamide, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N-(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentacarbonylchromium- or tungsten)carbonyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, 3-nitropyridinesulfenamide (Npys), p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

In certain embodiments, the processes described herein provided regio-selective azidation for B29-lysine in preference to the two N-amino termini A1-glycine and B1-phenyl alanine lysine present in RHI. This allowed diverse B29-triazole RHI derivatives to be synthesized by conjugating B29 azido RHI with various alkynes and are found to be potent human insulin receptor binders.

In certain embodiments the process described herein includes the following steps:
introducing RHI to a solvent system having a pH between 5-10;
adding an azotransfer agent to the RHI and the solvent system having a pH between 5-10; and
maintaining the environment at a pH between 5-10.

In certain embodiments the process described herein includes the following steps:
introducing RHI to a solvent system having a pH between 8-9;
adding an azotransfer agent; and
maintaining the environment at a pH between 8-9.

In the processes described herein, the azotransfer agent can be selected from imidazole-1-sulfonyl azide. HCl, BF₄, HSO₄, (2-azido-1-methyl-1H-imidazol-3-ium-3-yl)methanide compound with hexafluoro-16-phosphane, represented by the following formulas:

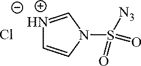 [SMILES: Cl ; [N-]=[N+]=NS(=O)(=O)n1ccnc1]

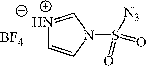 [SMILES: "BF4" ; [N-]=[N+]=NS(=O)(=O)n1ccnc1]

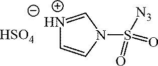 [SMILES: "HSO4" ; [N-]=[N+]=NS(=O)(=O)n1ccnc1]

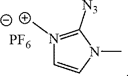

In the process described herein, the solvent system can comprise of any suitable solvent including, but not limited to, water, methanol (MeOH), isopropyl alcohol (iPrOH), tert- butanol (tBuOH), tert-amyl alcohol (tAmylOH) and dimethylacetamide (DMAc), or a combination thereof.

In certain embodiment, the solvent system of the processes described herein can be a combination of two or more solvents in optimal rations. Suitable ratios include, but are not limited to, are 9:1, 4:1, 7:3, 3:2, 1:1, 2:3, 3:7; 1:4 and 1:9.

In certain embodiments, the solvent system is a combination of methanol and water. A suitable ratio of water and methanol can include, but is not limited to, are 9:1, 4:1, 7:3, 3:2, 1:1, 2:3, 3:7; 1:4 and 1:9. In certain embodiments, the solvent system is a combination of water and methanol in a ratio of 9:1.

In certain embodiments, the solvent system is methanol. In certain embodiments, the solvent system is dimethylacetamide. In certain embodiments, the solvent system is isopropyl alcohol.

In the processes described herein the solvent system is at a pH between 5-10. In certain embodiments the solvent system used in the processes described herein is at a pH between 6-10. In certain embodiments the solvent system used in the processes described herein is at a pH between 7-10. In certain embodiments the solvent system used in the processes described herein is at a pH between 8-10. In certain embodiments the solvent system used in the processes described herein is at a pH between 9-10. In certain embodiments the solvent system used in the processes described herein is at a pH between 6-9. In certain embodiments the solvent system used in the processes described herein is at a pH between 6-8. In certain embodiments the solvent system used in the processes described herein is at a pH between 6-7. In certain embodiments the solvent system used in the processes described herein is at a pH between 8-9.

In certain embodiments the solvent system used in the processes described herein is at a pH of 6. In certain embodiments the solvent system used in the processes described herein is at a pH of 7. In certain embodiments the solvent system used in the processes described herein is at a pH of 8. In certain embodiments the solvent system used in the processes described herein is at a pH of 9. In certain embodiments the solvent system used in the processes described herein is at a pH of 10.

The solvent system can be adjusted or kept at the desired pH by using pH buffers. Buffers can be added at the beginning of the reaction or during the reaction, as needed, to keep the pH of the system at the desired pH. Suitable buffers include, but are not limited to, sodium bicarbonate, sodium potassium phosphate, sodium acetate, disodium phosphate, (NaHPO₄/Na2H₂PO₄) or sodium bicarbonate/sodium carbonate (Na₂CO₃/NaHCO₃).

In certain embodiments of the processes described herein, the buffered solvent system can also include copper (II) sulfate.5H₂0. In certain embodiments of the processes described herein, the buffered solvent system does not include copper (II) sulfate.5H₂0.

In certain embodiments the process described herein includes the following steps:
introducing RHI to isopropyl alcohol and using NaHPO₄/Na2H₂PO₄ to adjust the pH to 9;
adding an azotransfer agent to the RHI, isopropyl alcohol and NaHPO₄/Na2H₂PO₄; and
maintaining the environment at a pH of 9.

### Methods for Making Triazole Conjugates of RHI

Also disclosed herein are processes of preparing triazole conjugates of RHI. In certain embodiments, the processes comprise reacting an azide insulin analog, such as the ones described herein, with an alkyne.

In certain embodiments, the processes for preparing triazoles conjugates of RHI include a process comprising: dissolving an azide insulin described herein in a suitable solvent; adding an alkyne; adding CuSO₄.5H₂O and an antioxidant; and mixing until the desired product is achieved.

In the process described herein, the solvent system can comprise of any suitable solvent including, but not limited to, water, DMSO, methanol (MeOH), isopropyl alcohol (iPrOH), tert-butanol (tBuOH), tert-amyl alcohol (tAmylOH) and dimethylacetamide (DMAc), or a combination thereof.

In certain embodiments of the processes described herein, suitable alkynes have the following formula:

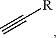

wherein R is selected from the group consisting of

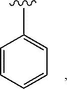

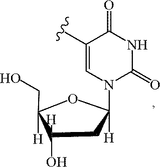

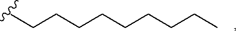

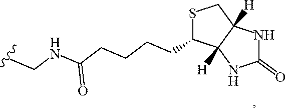

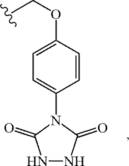

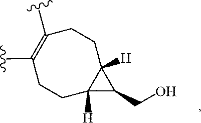

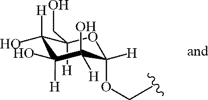

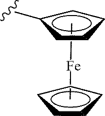

In certain embodiments of the process described herein, suitable antioxidants include mineral ascorbates, such as sodium ascorbate.

In certain embodiments of the processes described herein include the steps of, dissolving B29-N3 RHI in DMSO under nitrogen flow; adding an alkyne to the B29-N3 RHI in DMSO, adding CuSO₄.5H₂O in water to the B29-N3 RHI in DMSO; adding sodium ascorbate in water to the B29-N3 RHI in DMSO; stirring at room temperature; and adding 10 uL of 0.1 M HCl (aq).

### Methods of Treatment

The present invention provides a method for treating diabetes comprising administering to an individual with diabetes a therapeutically effective amount of a composition comprising an azide insulin analog or triazole conjugate of insulin described herein. In particular aspects the diabetes is Type 1 diabetes, Type 2 diabetes, or gestational diabetes.

The present invention provides for the use of a composition for the treatment of diabetes comprising an azide insulin analog or triazole conjugate of insulin described herein. In particular aspects the diabetes is Type 1 diabetes, Type 2 diabetes, or gestational diabetes.

The present invention provides for the use of an azide insulin analog or a triazole conjugate of insulin described herein for the manufacture of a medicament for the treatment of diabetes. In particular aspects the diabetes is Type 1 diabetes, Type 2 diabetes, or gestational diabetes.

The present invention provides a method for treating diabetes comprising forming an insulin dimer comprising an azide insulin analog described herein and administering to an individual with diabetes a therapeutically effective amount of a composition comprising the insulin dimer. In particular aspects the diabetes is Type 1 diabetes, Type 2 diabetes, or gestational diabetes.

The present invention provides for the use of a composition for the treatment of diabetes comprising an insulin dimer comprising an insulin analog described herein. In particular aspects the diabetes is Type 1 diabetes, Type 2 diabetes, or gestational diabetes.

The present invention provides for the use of an insulin dimer comprising insulin analog described herein for the manufacture of a medicament for the treatment of diabetes. In particular aspects the diabetes is Type 1 diabetes, Type 2 diabetes, or gestational diabetes.

### Azide Insulin Analogues Made in Accordance With the Processes Described Herein

Also described herein are the following compounds made using the processes described herein.

The present invention also provides azide insulin analogues selected from:

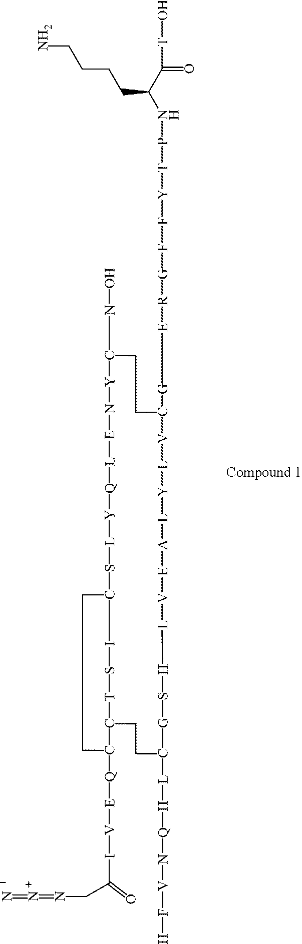

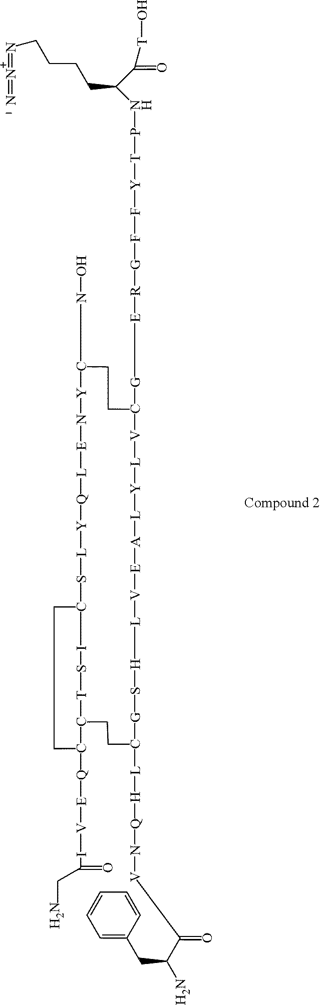

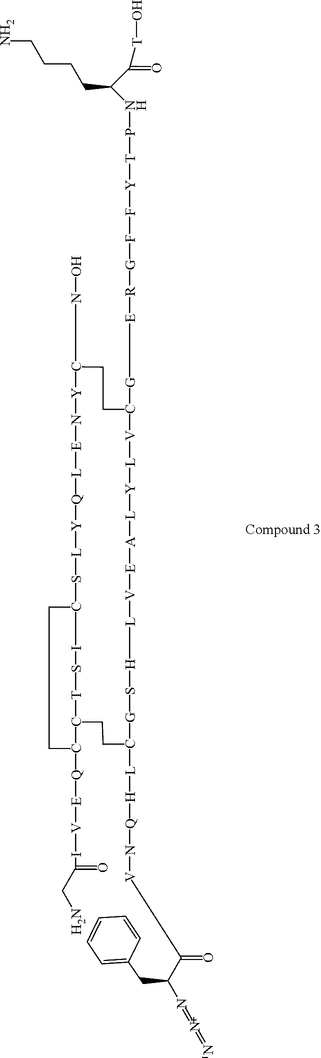

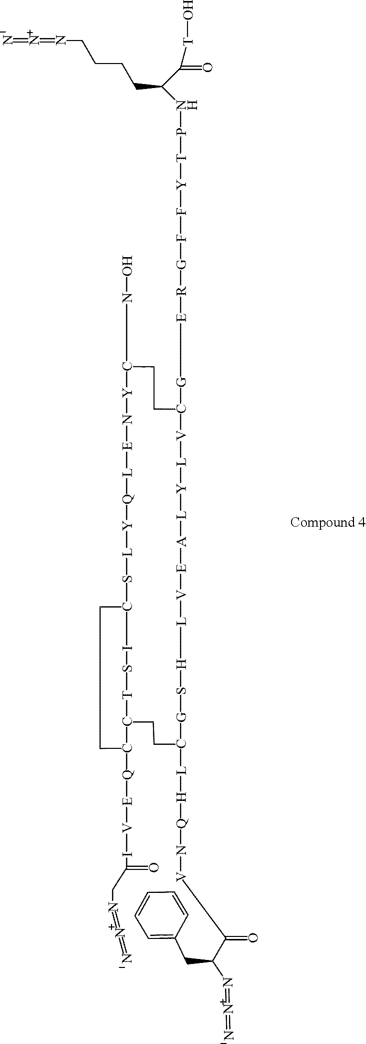

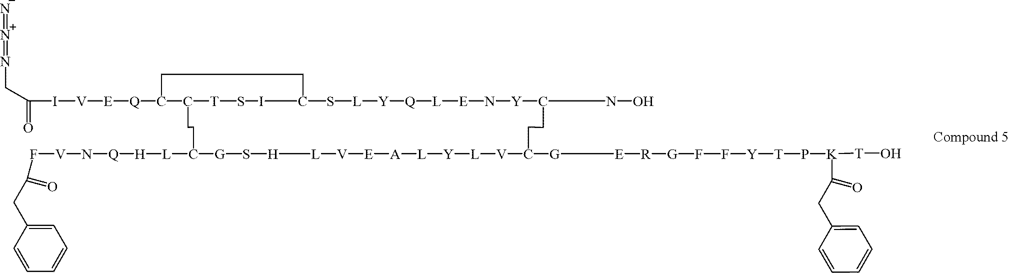

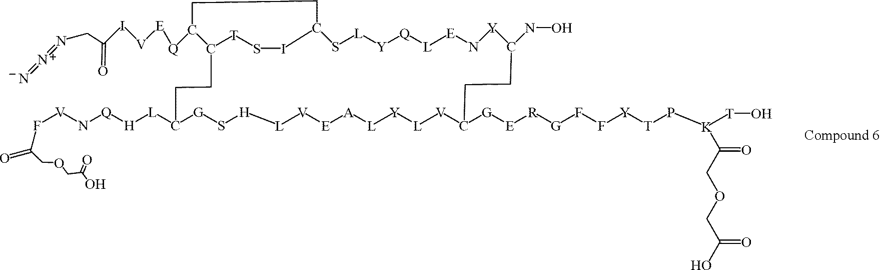

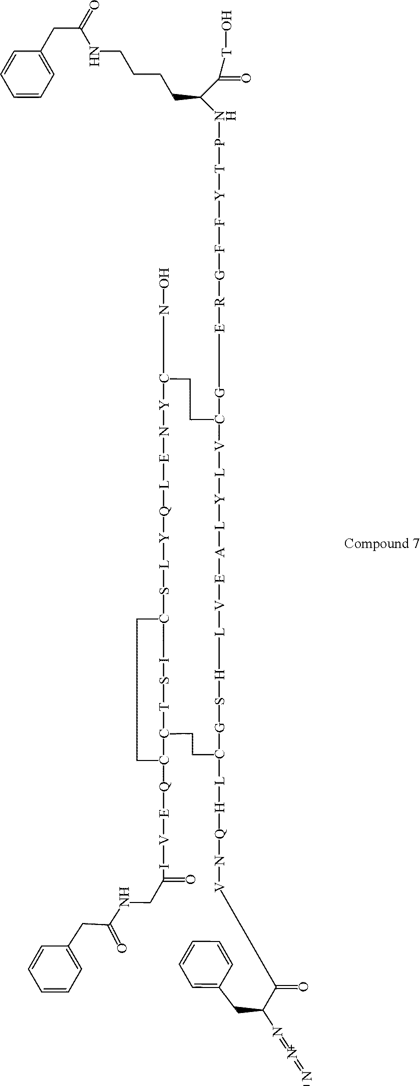

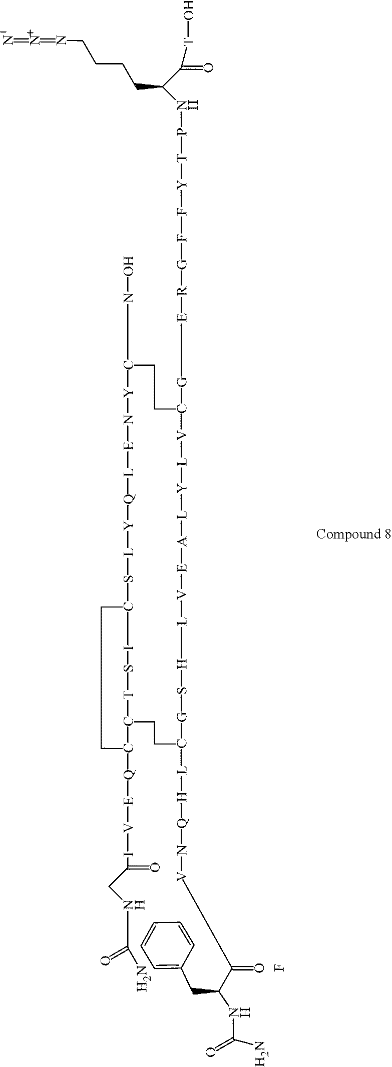

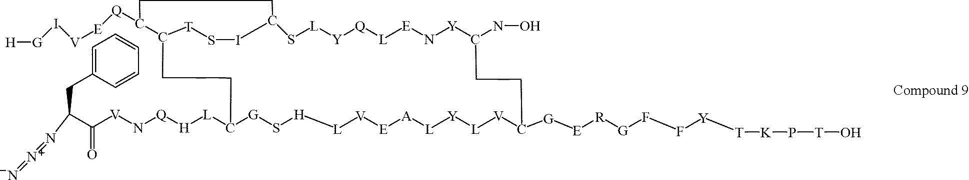

### Triazole Conjugates Made in Accordance With the Processes Described Herein

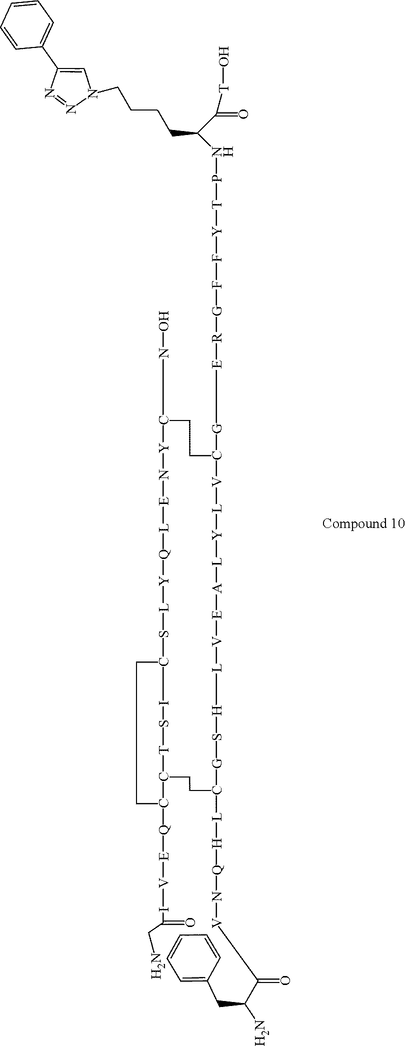

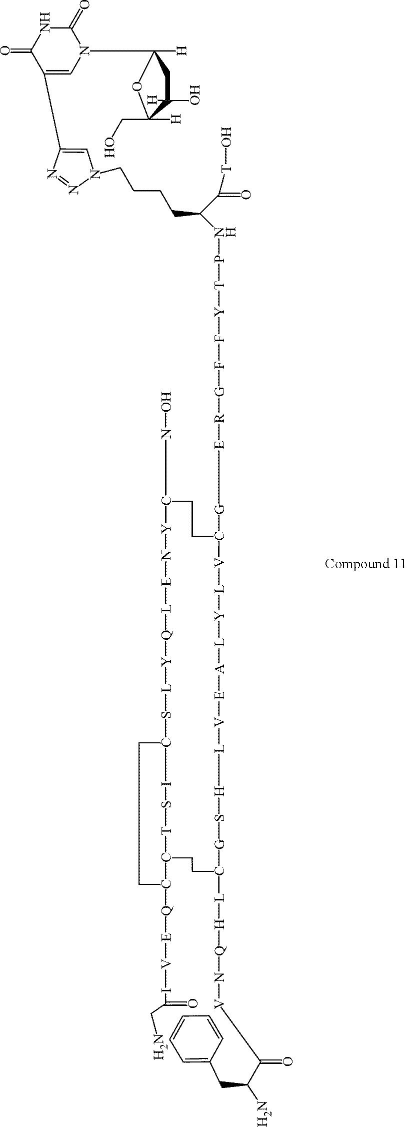

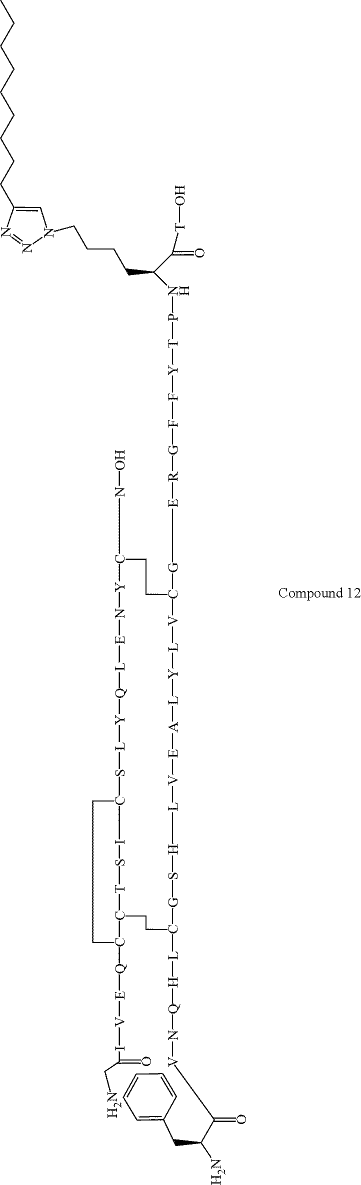

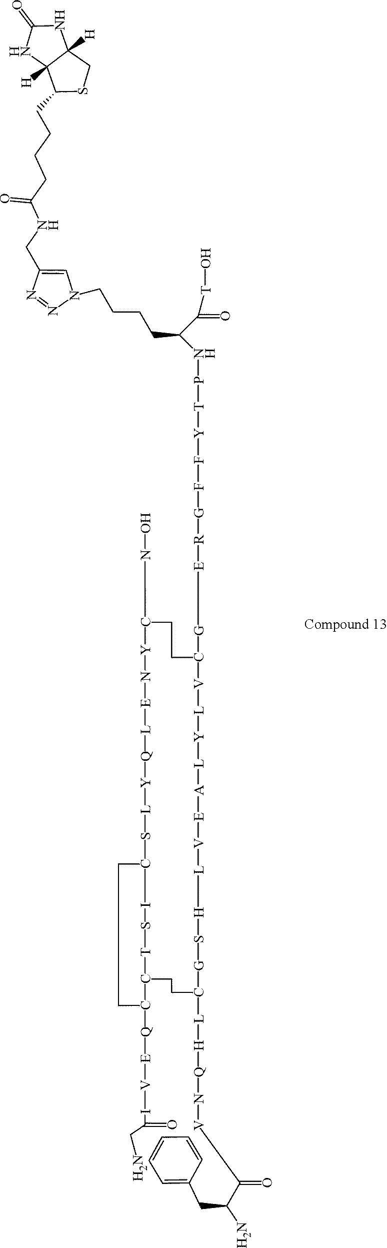

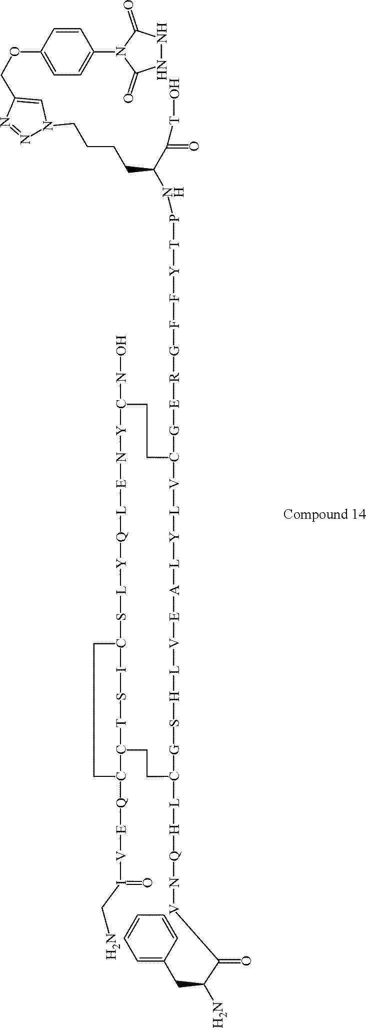

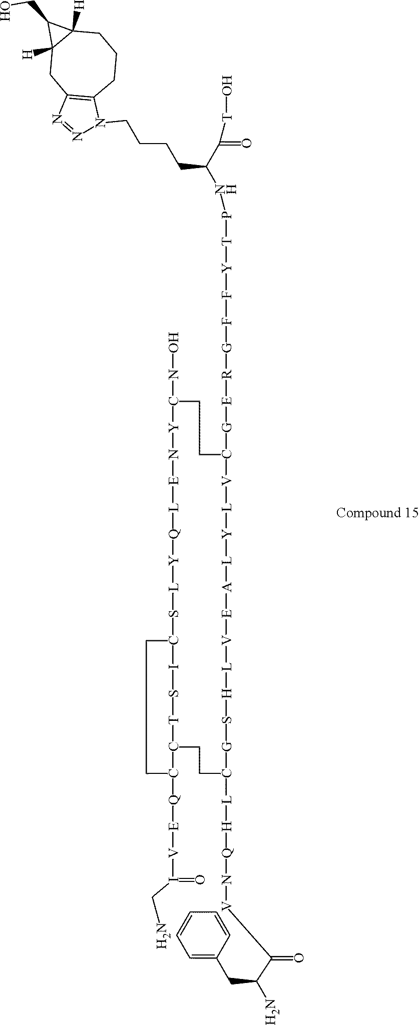

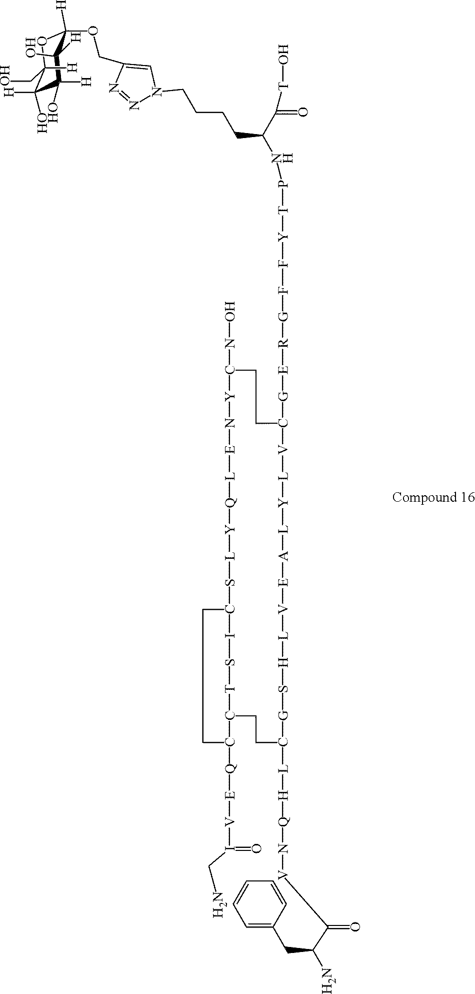

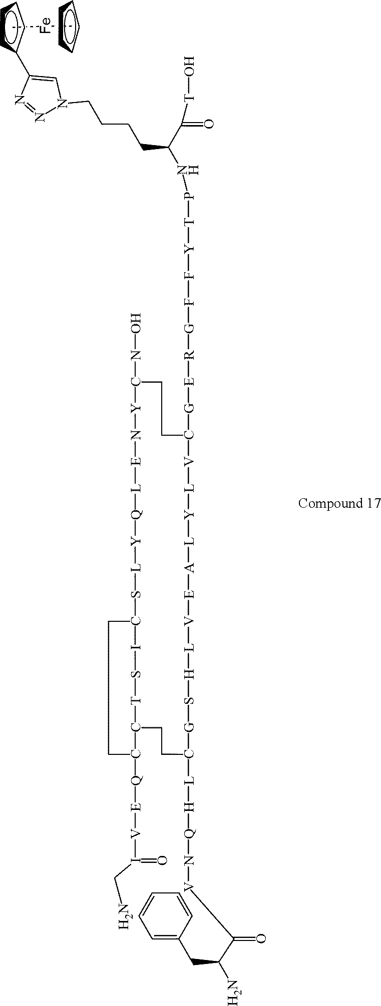

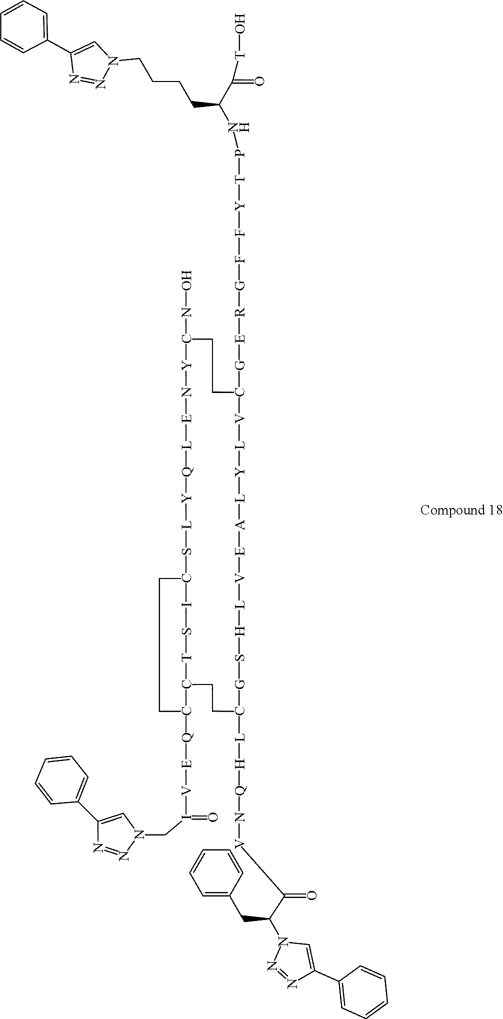

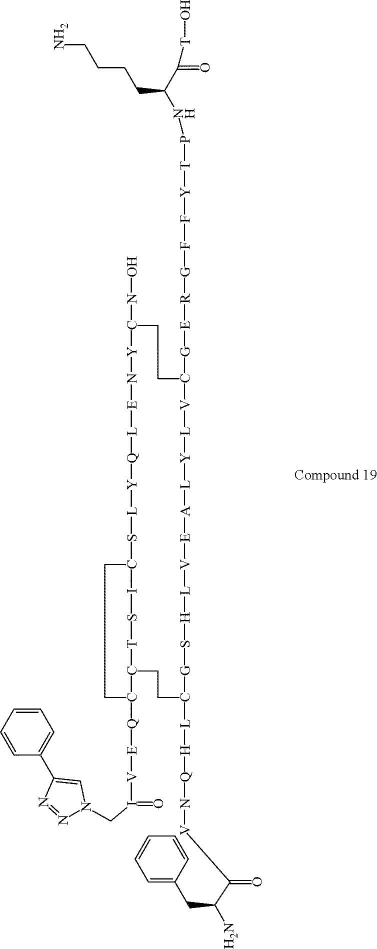

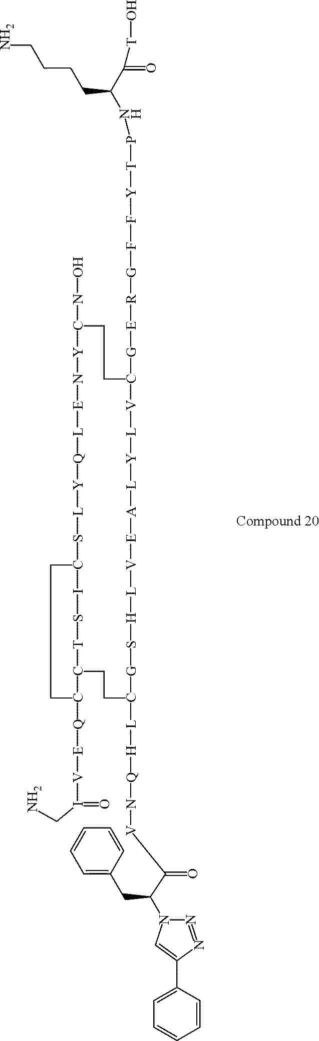

### Pharmaceutical Compositions

In accordance with one embodiment a pharmaceutical composition is provided comprising any of the azide insulin analogues or triazole conjugates of insulin described herein or insulin analog dimers comprising an azide insulin analog described herein, preferably at a purity level of at least 90%, 91%, 92%, 93%, 94%, 95%, 96%, 97%, 98% or 99%, and a pharmaceutically acceptable diluent, carrier or excipient. Such compositions may contain an azide insulin analog or triazole conjugate of insulin described herein or insulin analog dimers comprising an azide insulin analog described herein as disclosed herein at a concentration of at least 0.5 mg/ml, 1 mg/ml, 2 mg/ml, 3 mg/ml, 4 mg/ml, 5 mg/ml, 6 mg/ml, 7 mg/ml, 8 mg/ml, 9 mg/ml, 10 mg/ml, 11 mg/ml, 12 mg/ml, 13 mg/ml, 14 mg/ml, 15 mg/ml, 16 mg/ml, 17 mg/ml, 18 mg/ml, 19 mg/ml, 20 mg/ml, 21 mg/ml, 22 mg/ml, 23 mg/ml, 24 mg/ml, 25 mg/ml or higher. In one embodiment the pharmaceutical compositions comprise aqueous solutions that are sterilized and optionally stored contained within various package containers. In other embodiments the pharmaceutical compositions comprise a lyophilized powder. The pharmaceutical compositions can be further packaged as part of a kit that includes a disposable device for administering the composition to a patient. The containers or kits may be labeled for storage at ambient room temperature or at refrigerated temperature.

The disclosed azide insulin analogues or triazole conjugates of insulin described herein or insulin analog dimers comprising an azide insulin analog described herein are believed to be suitable for any use that has previously been described for insulin peptides. Accordingly, the azide insulin analogues or triazole conjugates of insulin described herein or insulin analog dimers comprising an azide insulin analog described herein disclosed herein can be used to treat hyperglycemia, or treat other metabolic diseases that result from high blood glucose levels. Accordingly, the present invention encompasses pharmaceutical compositions comprising an azide insulin analog or triazole conjugate of insulin described herein or insulin analog dimers comprising an azide insulin analog described herein as disclosed herein and a pharmaceutically acceptable carrier for use in treating a patient suffering from high blood glucose levels. In accordance with one embodiment the patient to be treated using an azide insulin analog or triazole conjugate of insulin described herein or insulin analog dimers comprising an azide insulin analog described herein is a domesticated animal, and in another embodiment the patient to be treated is a human.

One method of treating hyperglycemia in accordance with the present disclosure comprises the steps of administering the presently disclosed azide insulin analogues or triazole conjugates of insulin described herein or insulin analog dimers comprising an azide insulin analog described herein or insulin analog dimers to a patient using any standard route of administration, including parenterally, such as intravenously, intraperitoneally, subcutaneously or intramuscularly, intrathecally, transdermally, rectally, orally, nasally or by inhalation. In one embodiment the composition is administered subcutaneously or intramuscularly. In one embodiment, the composition is administered parenterally and the azide insulin analogues described herein or insulin analog dimers comprising an azide insulin analog described herein is prepackaged in a syringe.

The azide insulin analogues or triazole conjugates of insulin described herein or insulin analog dimers comprising an azide insulin analog or triazole conjugate of insulin described herein may be administered alone or in combination with other anti-diabetic agents. Anti-diabetic agents known in the art or under investigation include native insulin, native glucagon and functional analogues thereof, sulfonylureas, such as tolbutamide (Orinase), acetohexamide (Dymelor), tolazamide (Tolinase), chlorpropamide (Diabinese), glipizide (Glucotrol), glyburide (Diabeta, Micronase, Glynase), glimepiride (Amaryl), or gliclazide (Diamicron); meglitinides, such as repaglinide (Prandin) or nateglinide (Starlix); biguanides such as metformin (Glucophage) or phenformin; thiazolidinediones such as rosiglitazone (Avandia), pioglitazone (Actos), or troglitazone (Rezulin), or other PPARγ inhibitors; alpha glucosidase inhibitors that inhibit carbohydrate digestion, such as miglitol (Glyset), acarbose (Precose/Glucobay); exenatide (Byetta) or pramlintide; Dipeptidyl peptidase-4 (DPP-4) inhibitors such as sitagliptin, vildagliptin, saxagliptin, linagliptin, gemigliptin, anagliptin, teneligliptin, alogliptin, trelagliptin, omarigliptin, evogliptin, gosogliptin and dutogliptin; SGLT2 (sodium-dependent glucose transporter) inhibitors such as dapagliflozin, empagliflozin, canagliflozin, ipragliflozin, tofogliflozin, sergliflozin, remogliflozin, ertugliflozin and sotagliflozin; or FBPase (fructose 1,6-bisphosphatase) inhibitors.

Pharmaceutical compositions comprising the azide insulin analogues or triazole conjugates of insulin described herein or insulin analog dimers comprising an azide insulin analog described herein can be formulated and administered to patients using standard pharmaceutically acceptable carriers and routes of administration known to those skilled in the art. Accordingly, the present disclosure also encompasses pharmaceutical compositions comprising one or more of the azide insulin analogues or triazole conjugates of insulin described herein or insulin analog dimers comprising an azide insulin analog described herein, or a pharmaceutically acceptable salt thereof, in combination with a pharmaceutically acceptable carrier. For example, the pharmaceutical compositions comprising the azide insulin analogues or triazole conjugates of insulin described herein or insulin analog dimers comprising an azide insulin analog described herein may optionally contain zinc ions, preservatives (e.g., phenol, cresol, parabens), isotonicizing agents (e.g., mannitol, sorbitol, lactose, dextrose, trehalose, sodium chloride, glycerol), buffer substances, salts, acids and alkalis and also further excipients. These substances can in each case be present individually or alternatively as mixtures. Glycerol, dextrose, lactose, sorbitol and mannitol are customarily present in the pharmaceutical preparation in a concentration of 100-250 mM, NaCl in a concentration of up to 150 mM. Buffer substances, such as, for example, phosphate, acetate, citrate, arginine, glycylglycine or TRIS (i.e. 2-amino-2-hydroxymethyl-1,3-propanediol) buffer and corresponding salts, are present in a concentration of 5-250 mM, commonly from about 10-100 mM. Further excipients can be, inter alia, salts or arginine.

In one embodiment the pharmaceutical composition comprises a 1 mg/mL concentration of the azide insulin analogues or triazole conjugates of insulin described herein or insulin analog dimers comprising an azide insulin analog described herein at a pH of about 4.0 to about 7.0 in a phosphate buffer system. The pharmaceutical compositions may comprise the azide insulin analogues or triazole conjugates of insulin described herein or insulin analog dimers comprising an azide insulin analog described herein as the sole pharmaceutically active component, or the azide insulin analogues or triazole conjugates of insulin described herein or insulin analog dimers comprising an azide insulin analog described herein can be combined with one or more additional active agents.

All therapeutic methods, pharmaceutical compositions, kits and other similar embodiments described herein contemplate that azide insulin analogues or triazole conjugates of insulin described herein or insulin analog dimers comprising an azide insulin analog described herein include all pharmaceutically acceptable salts thereof.

In one embodiment the kit is provided with a device for administering the insulin analogues or triazole conjugates of insulin described herein or insulin analog dimers composition to a patient. The kit may further include a variety of containers, e.g., vials, tubes, bottles, and the like. Preferably, the kits will also include instructions for use. In accordance with one embodiment the device of the kit is an aerosol dispensing device, wherein the composition is prepackaged within the aerosol device. In another embodiment the kit comprises a syringe and a needle, and in one embodiment the insulin analogues described herein or insulin analog dimers composition is prepackaged within the syringe.

The following examples are intended to promote a further understanding of the present invention.

### EXAMPLES

Abbreviations: acetonitrile (ACN), aqueous (aq), dichloromethane (DCM), diisopropylethylamine (DIPEA), isopropyl acetate (IPAc), methyl t-butyl ether (MTBE), trifluoroacetic acid (TFA), mass spectrum (ms or MS), microgram(s) (µg), microliter(s) (µL), micromole (µmol), milligram(s) (mg), milliliter(s) (mL), millimole (mmol), minute(s) (min), phenyl (Ph), phenyl-3H-1,2,4-triazoline-3,5-(4H)dione (PTAD), dimethyl sulfoxide ( (DMSO) retention time (Rₜ), room temperature (rt), and trifluoroacetic acid (TFA).

The term “RHI” refers to recombinant human insulin and is used to indicate that the insulin has the amino acid sequence characteristic of native, wild-type human insulin. As used herein in the tables, the term indicates that the amino acid sequence of the insulin is that of native, wild-type human insulin.

### General Procedures

All chemicals were purchased from commercial sources, unless otherwise noted. Reactions were usually carried out at ambient temperature or at room temperature unless otherwise noted. Reactions sensitive to moisture or air were performed under nitrogen or argon using anhydrous solvents and reagents. The progress of reactions was monitored by ultra-performance liquid chromatography-mass spectrometry (UPLC-MS). Ultra performance liquid chromatography (UPLC) was performed on a Waters Acquity™ UPLC^{®} system.

### UPLC-MS Methods

### Method A

System: Waters Acquity I-Class with SQD2 MS detector
Column: Waters XSelect Peptide CSH C-18, 2.1 × 100 mm, 2.5 micron particle size
Mobile Phase: Water w/ 0.05% trifluoroacetic acid (TFA) + acetonitrile (MeCN)
Flow Rate: 0.75 ml/min
Gradient: 5-50% MeCN over 8 min.
Runtime: 10 min
PDA UV monitoring 280 nm
MS conditions: Electrospray +, scanning 400-3000 amu

### Method B

System: Waters ACQUITY UPLC System Original with SQD MS detector
Column: Waters XSelect Peptide CSH C-18, 2.1 × 50 mm, 2.5 micron particle size
Mobile Phase: Water w/0.1% TFA and MeCN w/0.1% TFA
Flow Rate: 1.0 ml/min
Gradient: 5-50% MeCN over 13 min.
Runtime: 15 min
PDA UV monitoring 215 nm
MS conditions: Electrospray +, scanning 125-2000 amu

### Method C

Waters Acquity™ UPLC^{®} BEH C8 1.7 µm 2.1×100 mm column with gradient 10:90-55:45 v/v CH₃CN/H₂O + v 0.05% TFA over 4.0 min and 55:45-95:5 v/v CH₃CN/H₂O + v 0.05% TFA over 40 sec; flow rate 0.3 mL/min, UV wavelength 200-300 nm; UPLC-MS;

Mass analysis was performed on a Waters SQ Detector with electrospray ionization in positive ion detection mode and the scan range of the mass-to-charge ratio was 170-900 or a Waters Micromass^{®} LCT Premier™ XE with electrospray ionization in positive ion detection mode and the scan range of the mass-to-charge ratio was 300-2000. The identification of the produced azide analogues was confirmed by comparing the theoretical molecular weight to the experimental value that was measured using UPLC-MS. For the determination of the linkage positions, specifically, insulin derivatives were subjected to DTT treatment (for a/b chain) or Glu-C digestion (with or without reduction and alkylation), and then the resulting peptides were analyzed by LC-MS. Based on the measured masses, the linkage positions were deduced.

Preparative scale HPLC was performed on Gilson 333-334 binary system using Waters DELTA PAK C4 15 µm, 300 Å, 50×250 mm column or KROMASIL^{®} C8 10 µm, 100 Å, 50×250 mm column, flow rate 85 mL/min, with gradient noted. Concentration of solutions was carried out on a rotary evaporator under reduced pressure or freeze-dried on a VirTis Freezemobile Freeze Dryer (SP Scientific).

### Synthesis of N^{2,1B},N^{6,29B}-bis oxyacetic acid RHI

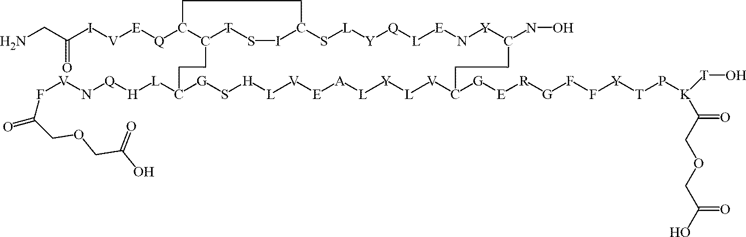

### Step 1 Synthesis of N^{2,1A}-Trifluoroacetyl -RHI:

DIPEA was added to a solution of RHI (1.0 g, 0.172 mmol) in DMSO (10.0 mL) 400 µl, 2.290 mmol) and the resulting mixture was stirred at room temperature for 5 minutes. Isopropyl 2,2,2-trifluoroacetate (150 µl, 1.065 mmol) was then added drop wise to the reaction mixture and the resulting mixture was stirred at room temperature for 2h. UPLC-MS Method C: Rt = 3.65 min, m/z = 1476.42 [(M+4)/4]. Crude reaction mixture was used in the following step without any purification.

### Step 2 B1/B29 Bis Oxyacetylation on N^{2,1A}-Trifluoroacetyl -RHI

A solution of 1,4-dioxane-2,6-dione (40 mg, 0.345 mmol) in 100 µL DMSO was added to the reaction mixture from Step 1. and the resulting mixture was stirred at room temperature for 2 hours. The crude reaction mixture was added drop wise to a round-bottom flask containing 150 mL of IPAc/MTBE (4:1). The resulting white suspension was filtered and rinsed with (3× 50 mL of IPAc). The material was dried under high vacuum for 1h and used in the following step without any further purification. UPLC-MS Method C: Rt = 3.86 min, m/z = 1534.95 [(M+4)/4].

### Step 3 Deprotection of Trifluoroacetyl :

The crude product of Step 2 was dissolved in 5.0 mL of 10% acetonitrile in water, then 5.0 mL of commercial ammonium hydroxide (28% m/v) was added drop wise at 0° C., and the mixture was stirred at same temperature for 2 hours. Upon completion, the crude reaction mixture was concentrated to 5.0 mL using spin-dialysis on a 10 K MWCO membrane Amicon tube, and diafiltration was continued using 100 mL water (pH=3.00) to a final volume about 20 mL and purified by HPLC. (Kromasil C8 250×50 mm, 10 µm, 100 Å column; Buffer A:0.1% TFA in water; Buffer B: 0.1% TFA in AcCN). Fractions containing the title conjugate were combined and lyophilized to give the title product as a white solid. UPLC-MS method C: Rt = 3.68 min, m/z = 1510.64 [(M+4)/4].

### General Method for the Azidation of Recombinant Human Insulin (RHI) or Peptide

IH-imidazole-1-sulfonyl azide hydrochloride (102 mg, 0.485 mmoll) was added to Insulin RHI (3000 mg, 0.485 mmol), sodium bicarbonate (5.79 mg, 0.069 mmol) and copper(II)sulfate.5H₂0 ((0.619 ml, 0.097 mmol) dissolved in water (3.2 mL) and MeOH (0.8 mL). The reaction was maintained pH 9 by adding aqueous saturated NaHCO₃ solution (~0.5-1 mL) and stirred at room temperature for 6 hours. The reaction was monitored by LCMS for the formation of mono, di and tri azide products. Added 1 mL of acetonitrile, added slowly a few drops of aq. 1N HCl (0.5 -1 mL), filtered and then subjected to HPLC purification for separation of products.

The term “RHI” refers to recombinant human insulin and is used to indicate that the insulin has the amino acid sequence characteristic of native, wild-type human insulin.

TABLE 1

RHI Azides prepared by General Method

| Compd.# | Description | Rt (min) | (M+4)/4 | UPLC Method |
|---|---|---|---|---|
| 1 | RHI_A1_N₃ | 5.43 | 1459.12 | A |
| 2 | RHI_B29_N₃ | 5.62 | 1459.20 | A |
| 3 | RHI_B1_N₃ | 5.76 | 1459.09 | A |
| 4 | RHI_A1_B1_B29_tri_N₃ | 8.60 | 1472.03 | A |
| 5 | RHI_A1_N₃_B1_B29_bis_PhAc | 9.4 | 1518.90 | B |
| 6 | RHI_A1_N₃_B1_B29_bis_MeOAcOH | 8.42 | 1517.66 | B |
| 7 | RHI_B1_N₃_A1_B29_bis_PhAc | 9.43 | 1518.74 | B |
| 8 | RHI_A1_B1_bis urea_B29_N₃ | 8.61 | 1480.58 | A |
| 9 | Lispro_B1_N₃ | 8.18 | 1460.07 | B |

### Insulin Receptor (IR) Binding Assays

IR binding assay was run in a scintillation proximity assay (SPA) in 384-well format using cell membranes prepared from CHO cells overexpressing human IR(B) grown in F12 media containing 10% FBS and antibiotics (G418, Penicillin/Strepavidin). Cell membranes were prepared in 50 mM Tris buffer, pH 7.8 containing 5 mM MgCl₂. The assay buffer contained 50 mM Tris buffer, pH 7.5, 150 mM NaCl, 1 mM CaCl₂, 5 mM MgCl₂, 0.1% BSA and protease inhibitors (Complete-Mini-Roche). Cell membranes were added to WGA PVT PEI SPA beads (5 mg/mL final concentration) followed by addition of insulin dimer molecules at appropriate concentrations. After 5-15 min incubation at room temperature,¹²⁵[I]-insulin was added at 0.015 nM final concentration for a final total volume of 50 µL. The mixture was incubated with shaking at room temperature for 1 to 12 hours followed by scintillation counting to determine ¹²⁵[I]-insulin binding to IR and the titration effects of insulin dimer molecules on this interaction.

### Insulin Receptor (IR) AKT-Phosphorylation Assays

Insulin receptor activation can be assessed by measuring phosphorylation of the Akt protein, a key step in the insulin receptor signaling cascade. CHO cell lines overexpressing human IR were utilized in an HTRF sandwich ELISA assay kit (Cisbio “Phospho-AKT(Ser473) and Phospho-AKT(Thr308) Cellular Assay Kits”). Cells were grown in F12 media supplemented with 10% FBS, 400 µg/mL G418 and 10 mM HEPES. Prior to assay, the cells were incubated in serum free media for 2 to 4 hr. Alternatively, the cells could be frozen and aliquoted ahead of time in media containing 20% DMSO and used in the assay upon thawing, spin down and re-suspension. Cells were plated at 10,000 cells per well in 20 µL of the serum free F12 media in 384-well plates. Humulin and insulin glargine controls were run on each plate of test compounds. The titrated compounds were added to the cells (2 µL per well, final concentrations = 1000 nM titrated down to 0.512 pM in 1:5 fold dilutions) and incubated at 37° C. for 30 min. The cells were lysed with 8 µL of the prepared lysis buffer provided in the CisBio kit and incubated at 25° C. for 1 hr. The diluted antibody reagents (anti-AKT-d2 and anti-pAKT-Eu3/cryptate) were prepared according to the kit instructions and then 10 µL was added to each well of cell lysate followed by incubation at 25° C. for 3.5 to 5 hr. The plate was read by in an Envision plate reader (Excitation = 320 nm; Emission = 665 nm) to determine the IR pAkt agonist activity with regard to both potency and maximum response for each compound. Alternatively, the compounds were tested in the same manner in the presence of 1.6 nM of Humulin to determine how each compound was able to compete against the full agonist activity of insulin.

TABLE 2

In Vitro Biological Activity

| Compd. # | IR Binding IC₅₀ (nM) | IR pAkt EC₅₀ (nM) | IR pAkt %Max |
|---|---|---|---|
| 1 | 3.8 | 0.26 | 91 |
| 2 | 1.2 | 0.21 | 101 |
| 3 | 4.7 | 0.14 | 103 |
| 4 | 5.3 | 0.19 | 89 |
| 5 | 5.1 | 0.17 | 98 |
| 6 | 9.8 | 0.23 | 89 |
| 7 | 10.2 | 0.51 | 94 |
| 8 | 7.4 | 0.23 | 88 |
| 9 | 2.5 | 0.06 | 89 |

### Site-Selective Azidation of RHI

### General Procedures

All chemicals were purchased from commercial sources, unless otherwise noted. Reactions were usually carried out at ambient temperature or at room temperature unless otherwise noted. Reactions sensitive to moisture or air were performed under nitrogen or argon using anhydrous solvents and reagents. The progress of reactions was monitored by ultraperformance liquid chromatography-mass spectrometry (UPLC-MS). Ultra performance liquid chromatography (UPLC) was performed on a Waters Acquity™ UPLC® system.

### UPLC-MS Method

System: Waters Acquity I-Class with SQD2 MS detector
Column: Waters XSelect Peptide CSH C-18, 2.1 × 100 mm, 2.5 micron particle size
Mobile Phase: Water w/ 0.05% trifluoroacetic acid (TFA) + acetonitrile (MeCN)
Flow Rate: 0.75 ml/min
Gradient: 5-50% MeCN over 8 min.
Runtime: 10 min
PDA UV monitoring 280 nm
MS conditions: Electrospray +, scanning 400-3000 amu

### Analytical Method

Data acquisition: Waters Synapt G2-Si, TOF resolution mode
HRMS condition:
ESI positive ion mode, range 100-3000 Da, scan time 0.3 sec, continuum mode
Source capillary 3 kV; Sampling cone 35 V; Source offset 35 V
Source temperature 150 C; Desolvation temperature 550 C
Cone gas 0 L/h; Desolvation gas 800 L/h; Nebuliser gas 6 bar
LC condition:
ACQUITY UPLC BEH C18 Column (130 Å, 1.7 µm, 2.1 mm × 100 mm) at 30 C
MPA: Water 0.05% TFA; MPB: Acetonitrile 0.05% TFA
0-0.5 min: 5% B; 0.5-5 min: 5-95% B; 5-6 min: hold at 95% B; 6-6.1 min: 95-5% B; 6.1-8 min:
5% B
Data processing: UNIFI, MaxEnt3
Input m/z range: 500-3000 Da
Output mass range: 4000-10000 Da
Maximum charge: 8
Width model: TOF (Resolution 30k)

Mass analysis was performed on a Waters SQ Detector with electrospray ionization in positive ion detection mode and the scan range of the mass-to-charge ratio was 170-900 or a Waters Micromass® LCT Premier™ XE with electrospray ionization in positive ion detection mode and the scan range of the mass-to-charge ratio was 300-2000. The identification of the produced azide analogs was confirmed by comparing the theoretical molecular weight to the experimental value that was measured using UPLC-MS. For the determination of the linkage positions, specifically, insulin derivatives were subjected to DTT treatment (for a/b chain) or Glu-C digestion (with or without reduction and alkylation), and then the resulting peptides were analyzed by LC-MS. Based on the measured masses, the linkage positions were deduced. Preparative scale HPLC was performed on Gilson 333-334 binary system using Waters DELTA PAK C4 15 µm, 300 Å, 50×250 mm column or KROMASIL® C8 10 µm, 100 Å, 50×250 mm column, flow rate 85 mL/min, with gradient noted. Concentration of solutions was carried out on a rotary evaporator under reduced pressure or freeze-dried on a VirTis Freezemobile Freeze Dryer (SP Scientific).
Data acquisition: Waters Synapt G2-Si, TOF resolution mode
HRMS condition:
ESI positive ion mode, range 100-3000 Da, scan time 0.3 sec, continuum mode
Source capillary 3 kV; Sampling cone 35 V; Source offset 35 V
Source temperature 150 C; Desolvation temperature 550 C
Cone gas 0 L/h; Desolvation gas 800 L/h; Nebuliser gas 6 bar
LC condition:
ACQUITY UPLC BEH C18 Column (130 Å, 1.7 µm, 2.1 mm × 100 mm) at 30 C
MPA: Water 0.05% TFA; MPB: Acetonitrile 0.05% TFA
0-0.5 min: 5% B; 0.5-5 min: 5-95% B; 5-6 min: hold at 95% B; 6-6.1 min: 95-5% B; 6.1-8 min: 5% B
Data processing: UNIFI, MaxEnt3
Input m/z range: 500-3000 Da
Output mass range: 4000-10000 Da
Maximum charge: 8
Width model: TOF (Resolution 30k)

### Preparations of Different pH Buffer Solutions:

pH 5 (70 ml 0.2 M-NaOAc and 30 ml 0.2 M-HOAc mixed)
pH 6 (6.15 ml 0.2 M-Na2HPO4, 43.85 ml 0.2 M-NaH2PO4; diluted to 100 ml with H2O)
pH 7 (30.5 ml 0.2 M-Na2HPO4, 19.5 ml 0.2 M-NaH2PO4; diluted to 100 ml with H2O)
pH 8 (47.35 ml 0.2 M-Na2HPO4, 2.65 ml 0.2 M-NaH2PO4; diluted to 100 ml with H2O)
pH 9 (10 ml 0.1 M-Na2CO3 and 90 ml 0.1 M-Na2HCO3 mixed)
pH 10 (60 ml 0.1 M-Na2CO3 and 40 ml 0.1 M-Na2HCO3 mixed)

### Procedure for the Synthesis of Mono Azido Insulins A1, B1 and B29 Azido RHI

IH-imidazole-1-sulfonyl azide hydrochloride (102 mg, 0.485 mmoll) was added to RHI (3000 mg, 0.485 mmol), sodium bicarbonate (5.79 mg, 0.069 mmol) and copper(II)sulfate.5H2O ((0.619 ml, 0.097 mmol) dissolved in water (3.2 mL) and MeOH (0.8 mL). The reaction was maintained at pH ~9 by adding aqueous saturated NaHCO₃ solution (~0.5-1 mL) and stirred at room temperature for 6 hours. The reaction was monitored by LCMS for the formation of mono, di and tri azide products. A few drops of 1N aq. HCl (made homogeous solution) was added and the mixture was filtered and purified by reverse phase chmromatography to give the azido RHI products:
A1-glycine azide RHI (80 mg, 3% yeild); Rt = 2.64 min.; Molecular formula: C257H381N67O77S6; HRMS: Calculated protonated exact mass: 5830.6354; Observed protonated exact mass: 5830.6626.
B29-lysine azide RHI (900 mg, 32% yield); Rt = 2.70 min.; Molecular formula: C257H381N67O77S6; HRMS: Calculated protonated exact mass: 5830.6354; Observed protonated exact mass: 5830.65576.
B1-phenyl alanine azide RHI (50 mg, 2% yield); Rt = 2.73 min.; Molecular formula: C257H381N67O77S6; HRMS: Calculated protonated exact mass: 5830.6354; Observed protonated exact mass: 5830.66748
A1B1B29-tri-azide RHI (146 mg, 5% yield); Rt = 2.95 min.; Molecular formula:
C257H377N71O77S6; HRMS: Calculated (m/z+H) exact mass: 5882.61697; Observed (m/z+H) exact mass: 5882.65674

### Synthesis of A1B1B29-tri-azide RHI

RHI (100 mg, 0.017 mmol), sodium bicarbonate (5.79 mg, 0.069 mmol), CuSO₄. 5H₂O (0.550 mg, 3.44 µmol) were dissolved in water (0.800 ml) and MeOH (0.200 ml). 1H-imidazole-1-sulfonyl azide.HCl salt (14.44 mg, 0.069 mmol) was added and the pH was maintained at pH 8-9 by adding aq. sat. NaHCO₃ solution and stired at room temperature for 6 h. LCMS showed the formation of the A1B1B29-tri-azide RHI. A few drops of 1N aq. HCl (made homogeous solution) was added and the mixture was filtered and purified by reverse phase chmromatography to give A1B1B29-tri-azide RHI (73 mg, 72%yield).

### Synthesis of B29-lysine Azido RHI

RHI (100 mg, 0.017 mmol) was dissolved in DMA (0.4 ml) and pH10 buffer was added (10 mL). 1Himidazole-1-sulfonyl azide. HCl salt (0.361 ml, 0.017 mmol, 10 mg/mL in MeOH) was added and the reaction mixture was stirred at room temperature over night. LCMS showed product formation of B29-lysine azide RHI azide products. A few drops of 1N aq. HCl (made homogeous solution) was added and the mixture was filtered and purified by reverse phase chmromatography to give B29-lysine azide RHI 5 (24 mg, 34% yield).

### Synthesis of B1-phenyl Alanine Azide RHI

RHI (100 mg, 0.017 mmol) was dissolved in DMA (0.4 ml) and pH 8 buffer (10 mL) was added. 1H-imidazole-1-sulfonyl azide. HCl salt (0.361 ml, 0.017 mmol, 10 mg/mL in MeOH) was added and the reaction mixture was stirred at room temperature overnight. LCMS showed product formation of B1-phenyl alanine azide RHI 6 azide products. A few drops of 1N aq. HCl (made homogeous solution) was added and the mixture was filtered and purified by reverse phase chmromatography to give B1-phenyl alanine azide RHI 6 (16 mg, 30% yield).

### Structural Analysis of Insulin Azides

Chemicals and Reagents All solvents were LC-MS grade and were purchased from Fisher Scientific (Waltham, MA). Sequencing grade endoproteinase Glu—C was purchased from Promega.

UPLC-MS Analysis A Waters Acquity UPLC system coupled to a Waters Premier qTOF mass spectrometer was used. The mass spectrometer was operated in ESI positive mode. The m/z range was from 200 to 4000 for intact insulin analogues analysis, and m/z range was from 50 to 2000 for GluC digests analysis. 3-5 microliters of intact insulin solutions were analyzed on a Waters Acquity BEH C18 column (1.7 µm, 2.1 × 50 mm), and GluC digested insulin sample solutions were analyzed on a Waters Acquity BEH C18 column (1.7 µm, 2.1 × 150 mm). Both columns were maintained at a temperature of 35° C. A 2-eluent linear gradient system was employed with a flow rate of 0.3 mL/min. Mobile phase A included water with 0.1% formic acid, and mobile phase B included acetonitrile with 0.1% formic acid. For intact insulin analogues, the total LC run time was 12 min. The initial condition was 2% B for 0.5 min, 2-95% B from 0.5 to 9 min, and 95% B from 9 to 11 min, then B was dropped from 95% to 2% in 0.1 min, and 2% B was kept until 12 min for equilibration. For GluC digested peptides, the total run time was 26 min. The initial condition was 5-20% B for the first 3 min, 20-40% B from 3 to 15 min, 40-95% B from 15 to 20 min, and 95% B from 20 to 22 min, then B was dropped from 95% to 5% in 0.1 min, and 2% B was kept until 26 min for equilibration.

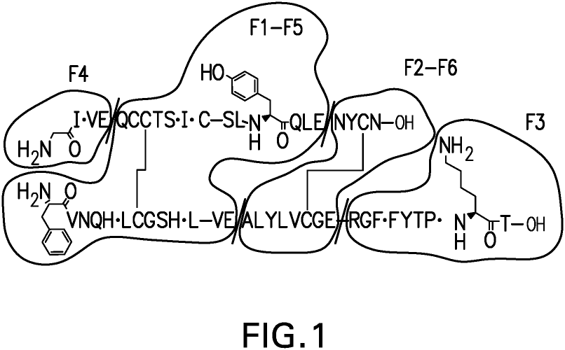

Results Firstly, the HPLC chromatogram and accurate mass were examined to check the purity and identity of the modified insulin analogues. The MaxEnt3 function in Waters MassLynx software was used to de-convolute the high-resolution mass spectrometry data. Afterwards, sequencing grade endoproteinase Glu—C was used to digest the modified insulin analogues overnight. Glu—C is a serine protease that specifically cleaves at the C-terminus of glutamic acid residues in ammonium bicarbonate buffer. The peptide digests generated were separated by UPLC, and the characteristic fragments were used to pinpoint the exact location of the azide modifications. For insulin, in FIG. 1, at least four peptide digests (labelled as F4, F1-F5, F2-F6 and F3) were generated after Glu—C digestion, and their expected exact masses were summarized in the table below. If N-terminus of the two insulin chains (A1, B1) or amino group of lysine (B29) is modified with azide, the expected exact mass is shifted by 26 Da respectively, with the mass values listed in the table. By comparing the LC/MS total ion chromatograms of the native human insulin and modified insulin analogues after Glu—C digestion separately, the peak(s) corresponding to peptide digests containing azide modification was identified.

TABLE 5

Expected Exact Masses

| GluC Fragments | Expected Exact Mass | |
|---|---|---|
| | Unmodified | N3 Modified |
| F4 | 416.2271 | 442.2271 (on A1) |
| F1-F5 | 2967.3024 | 2993.2929 (on B1) |
| F2-F6 | 1376.5741 | N/A |
| F3 | 1115.5764 | 1141.5669 (on B9) |

### General Method for the Preparation of Triazole Conjugates of Recombinant Human Insulin (RHI) or Insulin Analogs

B29-lysine azide RHI (1.714 µmol) was dissolved in DMSO (0.5 mL) under nitrogen flow, to this solution was added alkyne (3.43 µmol), added freshly prepared CuSO₄.5H₂O in water (5.14 µmol) followed by dropwise addition of Sodium ascorbate in water (6.86 µmol). Stirred the reaction mixture at room temperature for 1 h. LCMS showed product formation of B29-triazole RHI product. Added a few drops of 1N aq. HCl (made homogeous solution), filtered. Purification by reverse phase chmromatography gave B29-triazole RHI product.

Note: Compound 15, was prepared with Cu-free strain-promoted azide-alkyne cycloaddition (SPAAC) with no CuSO₄.5H₂O addition in the reaction.

### Analytical Method (X)

Data acquisition: Waters Synapt G2-Si, TOF resolution mode
HRMS condition:
   ESI positive ion mode, range 100-3000 Da, scan time 0.3 sec, continuum mode
   Source capillary 3 kV; Sampling cone 35 V; Source offset 35 V
   Source temperature 150 C; Desolvation temperature 550 C
   Cone gas 0 L/h; Desolvation gas 800 L/h; Nebuliser gas 6 bar
LC condition:
   ACQUITY UPLC BEH C18 Column (130 Å, 1.7 µm, 2.1 mm × 100 mm) at 30° C.
   MPA: Water 0.05% TFA; MPB: Acetonitrile 0.05% TFA
   0-0.5 min: 5% B; 0.5-5 min: 5-95% B; 5-6 min: hold at 95% B; 6-6.1 min:
   95-5% B; 6.1-8 min: 5% B
Data processing: UNIFI, MaxEnt3
   Input m/z range: 500-3000 Da
   Output mass range: 4000-10000 Da
   Maximum charge: 8
   Width model: TOF (Resolution 30k)

TABLE 3

Insulin triazole conjugates prepared by General Method

| Compd. # | Alkyne 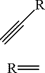 | Description | Mol. Formula | Rt (min) | HRMS (m/z+H) |
|---|---|---|---|---|---|
| 10 | 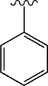 | RHI_B29_triazole_Phenyl acetylene | C₂₆₅H₃₈₇N₆₇O₇₇S₆ | 2.74 | 5932.70605 |
| 11 | 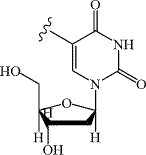 | RHI_B29_triazole_5-ethynyl-2′-deoxyuridine | C₂₆₈H₃₉₃N₆₉O₈₂S₆ | 2.59 | 6083.73486 |
| 12 | 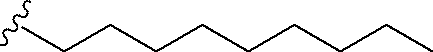 | RHI_B29_triazole_1-undecyne | C₂₆₈H₄₀₁N₆₇O₇₇S₆ | 3.1 | 5982.84277 |
| 13 | 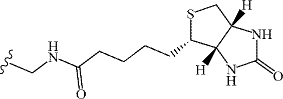 | RHI_B29_triazole_Biotin alkyne | C₂₇₀H₄₀₀N₇₀₀O₇₉S₇ | 2.6 | 6111.79785 |
| 14 | 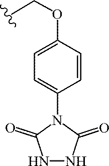 | RHI_B29_triazole_4-(4-(prop-2-yn-1-yloxy)phenyl)-1.2,4-triazolidine-3,5-dione | C₂₆₈H₃₉₀N₇₀O₈₀S₆ | 2.6 | 6062.73975 |
| 15 | 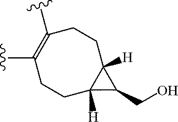 | RHI_B29_triazole_(1R,8S,9S)bicyclo[6.1]non-4-yn-9-ylmethanol | C₂₆₇H₃₉₅N₆₇O₇₈S₆ | 2.61 | 5980.75586 |
| 16 | 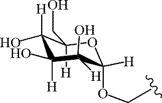 | RHI_B29_triazole_Propargyl-α-D-mannopyranoside | C₂₆₆H₃₉₅N₆₇O₈₃S₆ | 2.57 | 6049.73682 |
| 17 | 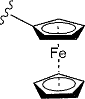 | RHI_B29_triazole_Ferrocene-1-alkyne | C₂₆₉H₃₉₃FeN₆₇O₇₇S₆ | 2.82 | 6040.67773 |
| 18 | 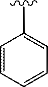 | RHI_A1B1B29_tritriazole_Phenyl acetylene | C₂₈₁H₃₉₅N₇₁O₇₇S₆ | 3.09 | 6188.81641 |
| 19 | 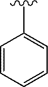 | RHI_A1_triazole_Phenyl acetylene | 3.18 | 148 | |
| 20 | 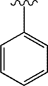 | RHI_B1_triazole_Phenyl acetylene | 3.28 | 148 | |

TABLE 4

In Vitro Biological Activity

| Compd. # | IR Binding IC₅₀ (nM) | IR pAkt EC₅₀ (nM) | IR pAkt %Max |
|---|---|---|---|
| 10 | 2.2 | 0.1 | 86 |
| 11 | 0.59 | 0.21 | 104 |
| 12 | 33 | 0.37 | 92 |
| 13 | 0.92 | 0.11 | 91 |
| 14 | 1.7 | 0.32 | 97 |
| 15 | 1.2 | 0.26 | 109 |
| 16 | 1.0 | 0.1 | 84 |
| 17 | 5.9 | 0.36 | 123 |
| 18 | 21 | 1.0 | 93 |
| 19 | 1.1 | 1.06 | |
| 20 | 0.33 | 0.32 | |

### Site-Selective Bio-Conjugation of A1B1B29 RHI

Site-selective bio-conjugation at each of the A1, B1 and B29 positions present in RHI 1 was explored. Treatment of ISA to B-29 phenyl triazole (Comound 10) at pH 8 with DMAc as co-solvent in the absence of Cu(II) (optimized condition for B1-selectivity) for two nights at ambient temperature proceeded smoothly giving rise to site-selective B1-azido B29-phenyltriazole RHI (Compound 21) as the major product in 66% isolated yield after purification. Facile copper catalyzed [2+3] cycloaddition reaction between Biotin propargyl alkyne and Compound 22 gave bis-triazole conjugated derivative, B1-Biotin-B29-phenyl triazole RHI Compound 23 in 55% isolated yield after purifi-cation. Treatment of reducing agent, Di-thio-Threotol (DTT) to Comound 23 in ammonium bicarbonate buffer at 56° C. for 30 min, led to the reduction of both S—S disulfide bonds and gave fragments with Biotin and phenyl triazole moieties on the B chain (m/z=3863.16309) and while A chain is unmodified (m/z=2382.57202). This provided the proof for the site-selective azidation on B1 in Compound 22. The RHI derivative Compound 23 with free A1-glycine amine free is poised for di-verse bio-conjugation reactions.

### Synthesis of B1-azido B29-phenyltriazole RHI Compound 21

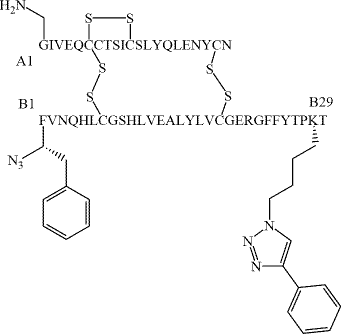

Compound 10 (B1-phenyl triazole RHI) (60 mg, 10.11 µmol) was dissolved in DMA (0.4 ml) and pH 8 buffer (10 mL) and 1H-imidazole-1-sulfonyl azide. HCl salt (10.11 µmol) was added. The reaction mixture was stirred at room temperature for 2 nights and monitored for the formation of the product by LCMS. A few drops of 1N aq. HCl was added and then the solution was filtered and purified by reverse phase chromatography to yield Comound 21. Rt = 3.40 min.; Molecular formula: C₂₆₅H₃₈₅N₆₉O₇₇S₆; HRMS: Calculated protonated exact mass: 5958.6729; Observed protonated exact mass: 5958.5850

### Synthesis of B1-Biotin Propargyl triazole-B29-phenyl Triazole RHI Compound 22

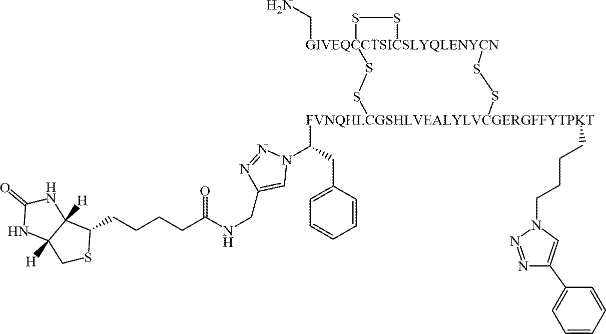

Compound 21(10 mg, 1.677 µmol) was dissolved in DMSO (0.5 mL) under nitrogen flow and to this solution 5-((3aS,4S,6aR)-2-oxohexahydro-1H-thieno[3,4-d]imidazol-4-yl)-N-(prop-2-yn-1-yl)pentanamide (3.35 µmol) and freshly prepared CuSO₄.5H₂O in water (5.03 µmol) was added followed by dropwise addition of sodium 2-(1,2-dihydroxy-ethyl)-4-hydroxy-5-oxo-2,5-dihydro-furan-3-olate dissolved in water (6.71 µmol). The reaction mixture was stirred at room temperature for 2 h and product formation was followed by LCMS. A few drops of 1N aq. HCl was added and the solution was filtered and purified by reverse phase chromatography to yield Compound 22. Rt = 3.30 min.; Molecular formula: C₂₇₈H₄₀₄N₇₂O₇₉S₇; HRMS: Calculated protonated exact mass: 6239.7927; Observed protonated exact mass: 6239.6421

### Synthesis of A1-methoxy PEG5 amide-B1-Biotin triazole-B29-phenyltriazole RHI Compound 23

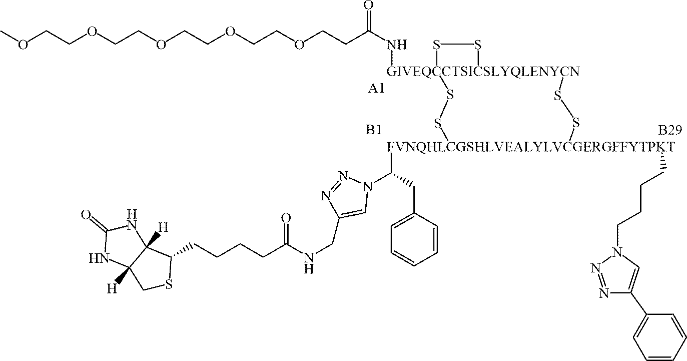

Compound 22 (3 mg, 0.481 µmol) was dissolved in acetonitrile and water (9:1, 1 mL) under nitrogen flow. To this solution triethyl amine (2.88 µmol) was added, followed by dropwise addition of methoxy-PEG5-NHS ester (0.961, µmol) dissolved in acetonitrile. The reaction mixture was stirred at room temperature for 2 h and product formation was followed by LCMS. A few drops of 1N aq. HCl was added and the solution was filtered and purified by reverse phase chromatography to give Compound 23. Rt = 3.30 min.; Molecular formula: C₂₉₀H₄₂₆N₇₂O₈₅S₇; HRMS: Calculated protonated exact mass: 6501.9343; Observed protonated exact mass: 6500.7217

TABLE 5

| Compd. No. | Description | Rt (min) | Exact Mass m/z |
|---|---|---|---|
| 21 | B1-azido B29-phenyltriazole RHI | 3.4 | 5958.6729 |
| 22 | B1-Biotin propargyl triazole-B29-phenyl triazole RHI | 3.30 | 6239.7927 |
| 22 | A1- Methoxy PEG5 amide, B1-biotin triazole, B29-phenyltriazole RHI | 3.1 | 1497.21 |

TABLE 6

In Vitro Biological Activity

| Compd. No. | IR Binding IC₅₀ (nM) |
|---|---|
| 21 | 1.2 |
| 22 | 0.74 |
| 23 | 6.0 |

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

What is claimed:

1. A process of making an azide insulin analog comprising steps of:
(a) providing an solvent system comprising an insulin or insulin analog thereof, sodium bicarbonate, Copper(II) sulfate, and 20% methanol at pH 9, wherein the insulin or analog thereof comprises an A chain having a free amine at the glycine at position A-1 and a B chain having free amines at the lysine at position B-29 and the phenyl alanine at position B-1;
(b) adding 1H-imidazole-1-sulfonyl azide hydrochloride to the solvent system;
(c) incubating the solvent system at room temperature for six hours while maintaining the solution at pH 9 to add the azide from the 1H-imidazole-1-sulfonyl azide hydrochloride to the insulin or insulin analog thereof; and
(d) adding acetonitrile to the solvent system and purifying the azide insulin analog to provide the azide insulin analog.

2. The process of claim 1, wherein the insulin is a recombinant human insulin.

3. The process of claim 1, wherein the free amine of the lysine at position B-29 is converted to an azide; the free amine of the glycine at position A-1 is converted to an azide; the free amine of the phenyl alanine at position B-1 is converted to an azide; the free amines of the lysine at position B-29, the glycine at position A-1 and the phenyl alanine at position B-1 are converted each to an azide; or, two of the three of the free amines of the lysine at position B-29, the glycine at position A-1, and the phenyl alanine at position B-1 are converted to an azide.

4. The process of claim 1, wherein the azide insulin analog is selected from: 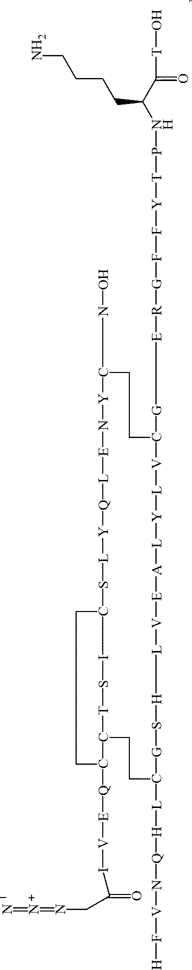 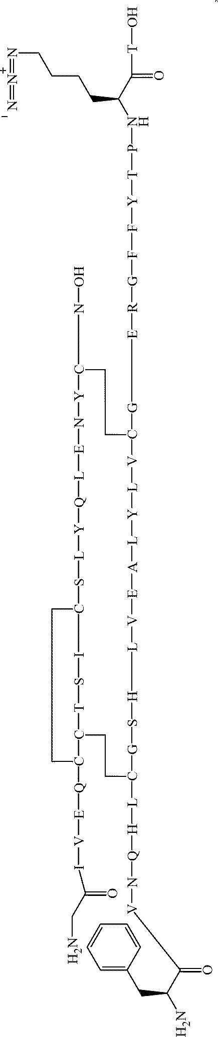 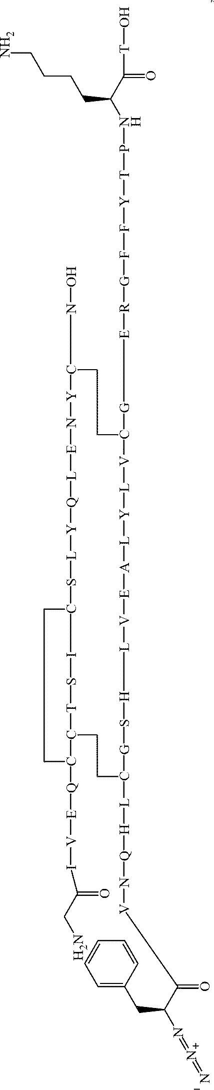 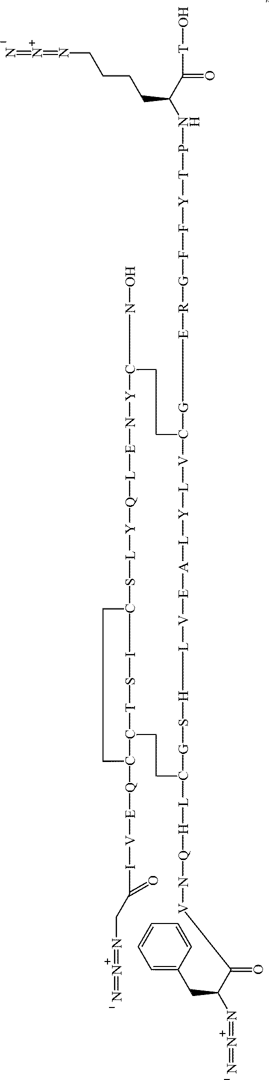 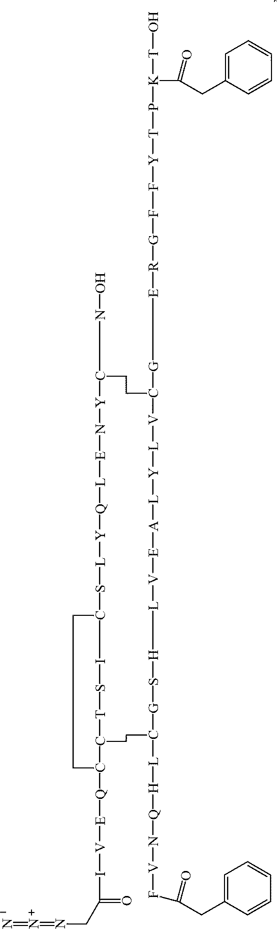 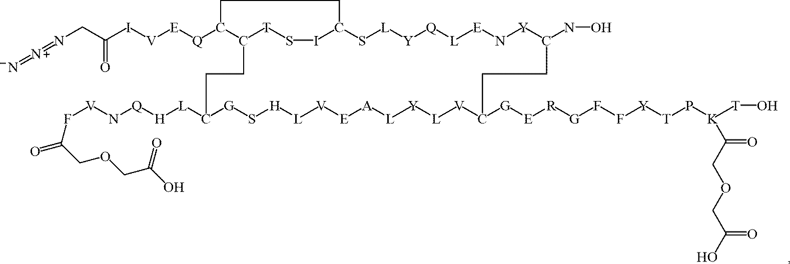 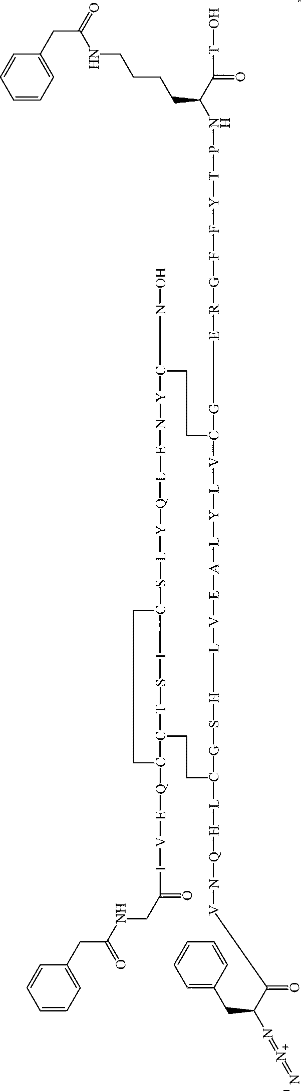 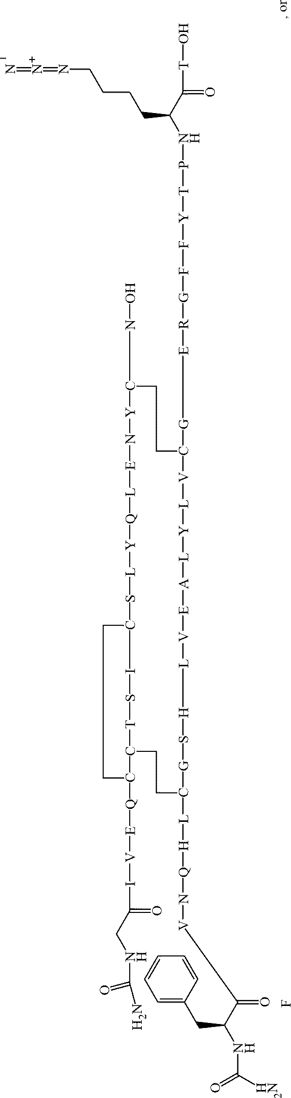 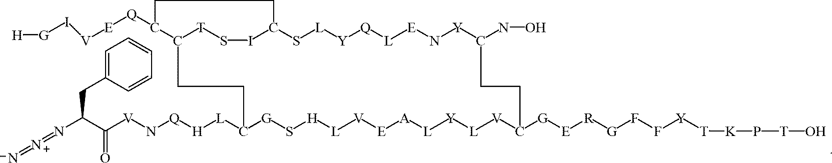
.

5. A process of making an azide insulin analog comprising:
(a) introducing an insulin or an insulin analog therof to a solvent system comprising a mixture of water and methanol and having a pH between 5-10, wherein the insulin or analog thereof comprises an A chain having a free amine at the glycine at position A-1 and a B chain having free amines at the lysine at position B-29 and the phenyl alanine at position B-1;
(b) adding an azotransfer agent to the solvent system comprising the insulin or insulin analog; and
(c) maintaining the solvent system comprising the azotransfer agent and the insulin or insulin analog at a pH between 5-10 to produce the azide inulin analog.

6. The process of claim 5, wherein the insulin is a recombinant human insulin.

7. The process of claim 5, wherein the azotransfer agent is 2-azido-1,3-dimethylimidazolinium PF6, imidazole-1-sulfonyl azide BF4, 1H-imidazole-1-sulfonyl azide HCl or 1H-imidazole-1-sulfonyl azide sulfate.

8. The process of claim 5, wherein sodium bicarbonate is further added to the solvent system.

9. The process of claim 5, wherein copper(II) sulfate.5H₂O is further added to the solvent system.

10. The process of claim 5, wherein the pH is maintained between 8-9.

11. The process of claim 5, wherein the lysine at position B-29 is converted to an azide; the free amine of the glycine at position A-1 is converted to an azide; the phenylalanine at position B-1 is converted to an azide; the lysine at position B-29, the glycine at position A-1 and the phenylalanine at position B-1 are converted each to an azide; or, two of the three of the free amines of the lysine at position B-29, the glycine at position A-1, and the phenylalanine at position B-1 are converted to an azide.

12. An azide insulin analog, or pharmaceutically acceptable saltt thereof, wherein the azide insulin analog is produced by the process of claim 1.

13. An azide insulin analog, or pharmaceutically acceptable salt thereof, wherein the azide insulin analog is produced by the process of claim 5.

14. A compound, or pharmaceutically acceptable salt thereof, selected from: 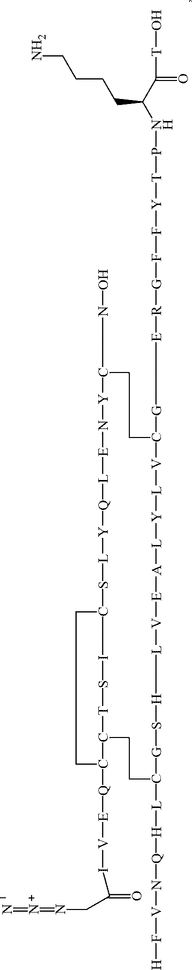 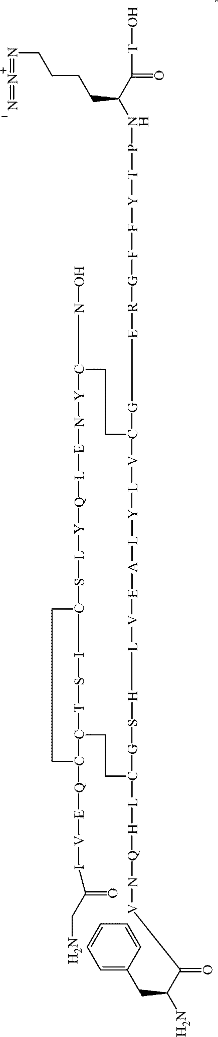 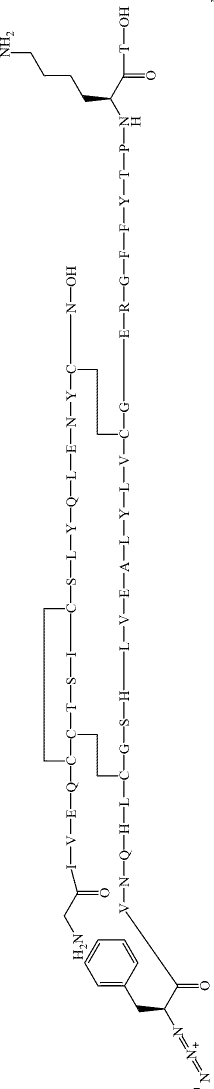 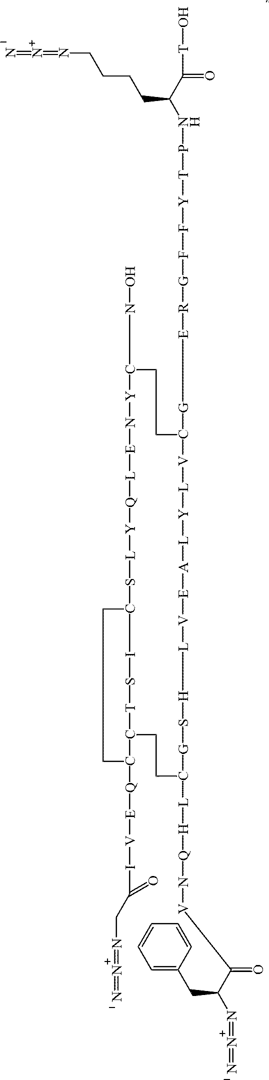 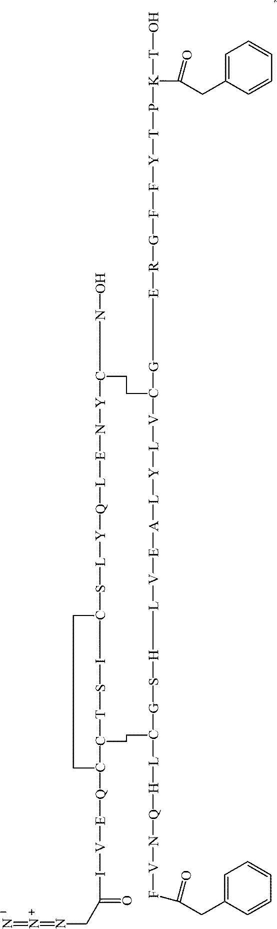 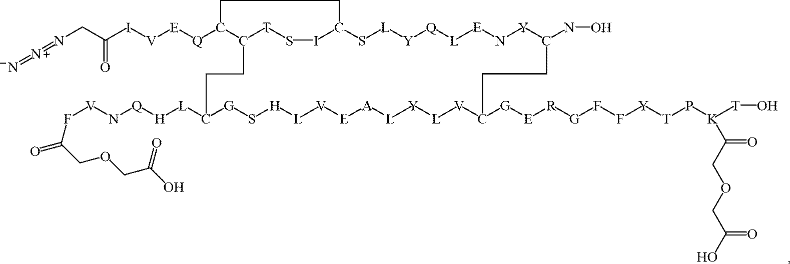 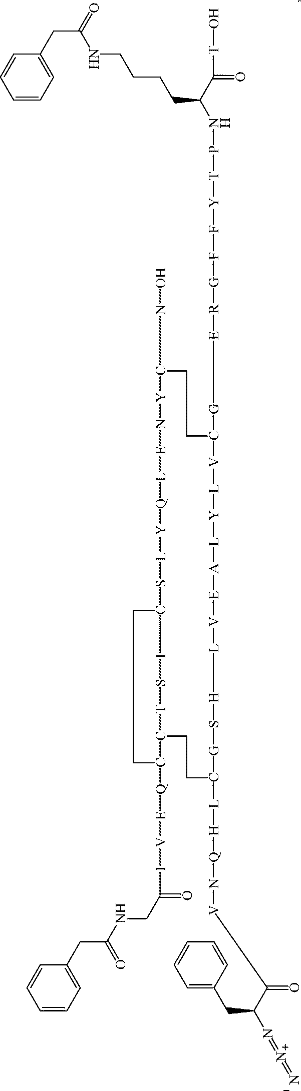 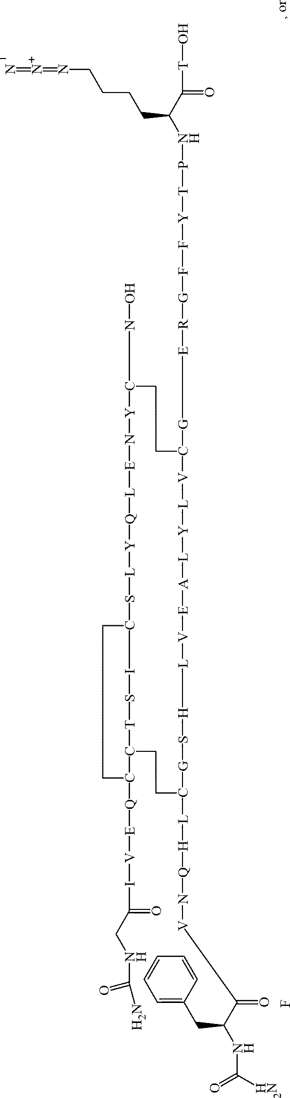 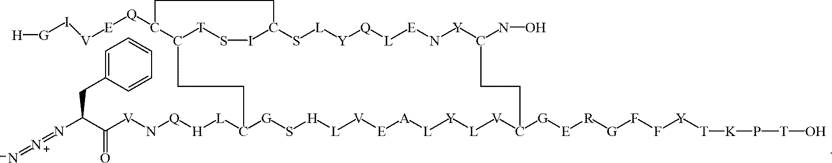
.

15. A process for preparing a triazole conjugate of an insulin or insulin analog thereof comprising reacting an azide insulin analog produced by the process of claim 1 with an alkyne to produce the triazole conjugate of the insulin or insulin analog thereof.

16. The process of claim 15, wherein the alkyne has the following formula 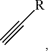
wherein R is selected from the group consisting of 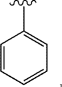 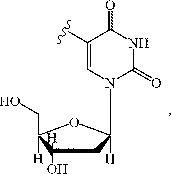 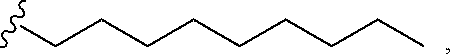 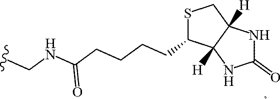 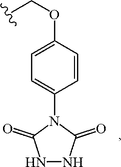 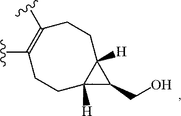 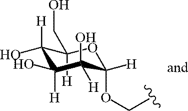 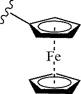
.

17. A triazole conjugate of an insulin or insulin analog thereof, or pharmaceutically acceptable salt thereof, wherein the triazole conjugate of the insulin or insulin analog is produced by the process of claim 15.

18. The process of claim 17, wherein the alkyne has the following formula 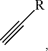
wherein R is selected from the group consisting of 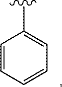 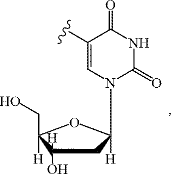 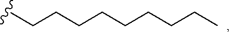 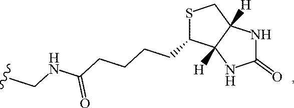 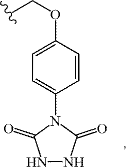 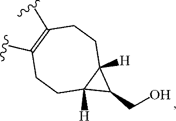 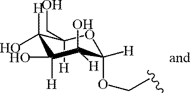 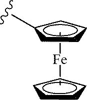
.

19. A compound, or pharmaceutically acceptable salt thereof, selected from: 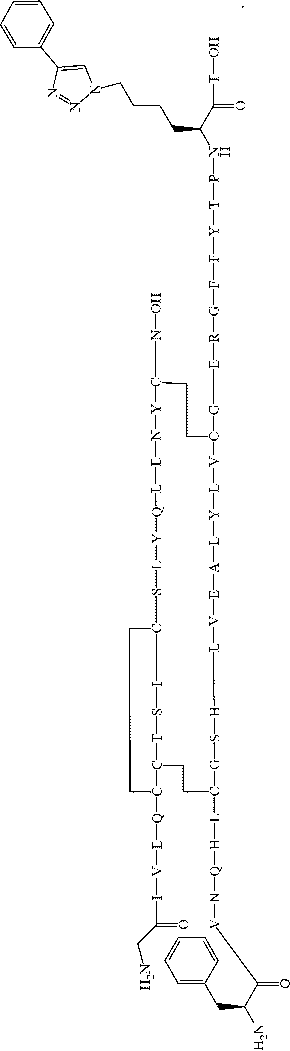 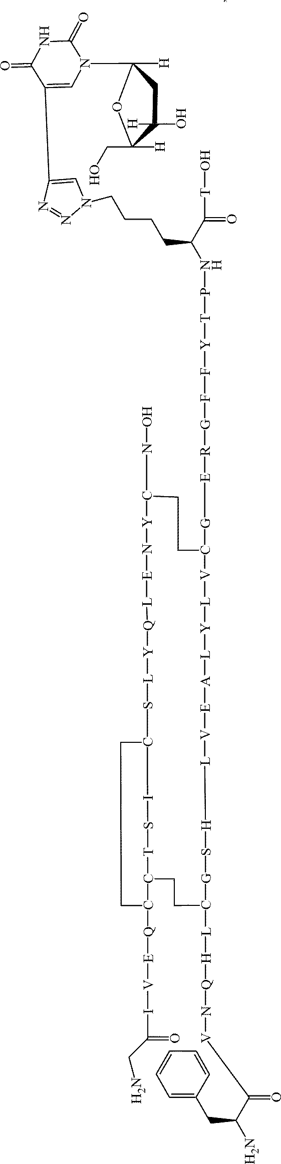 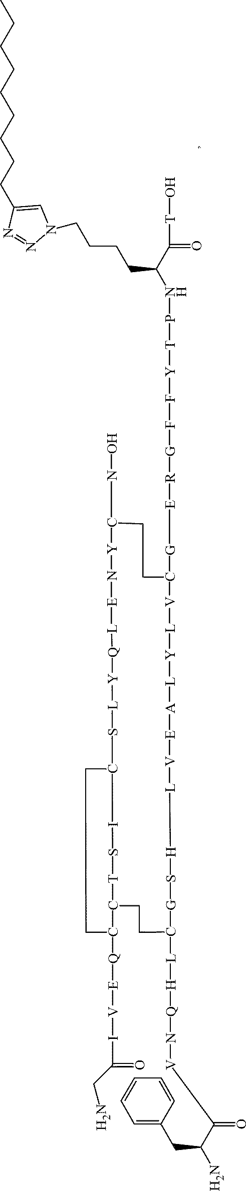 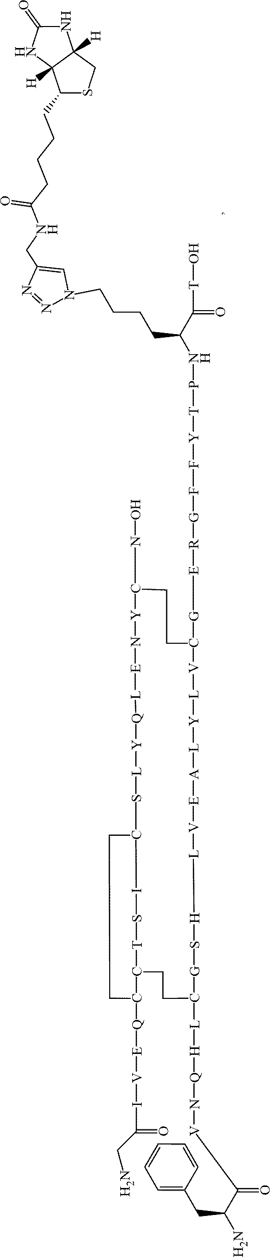 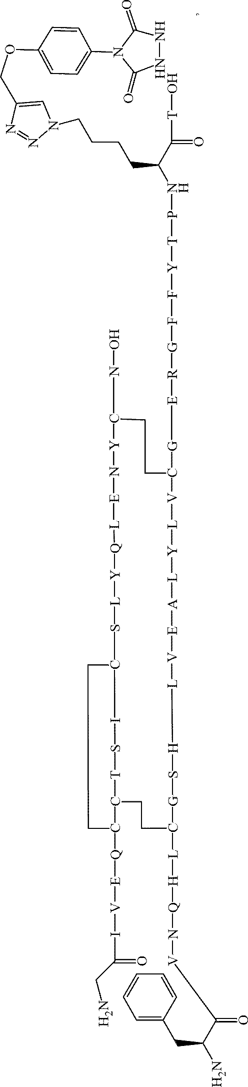 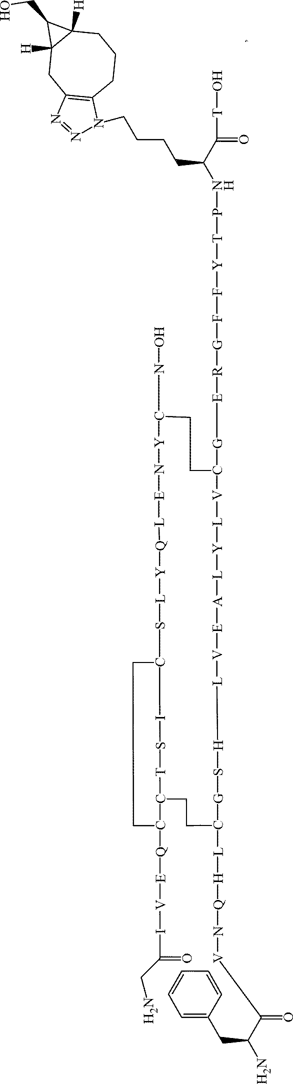 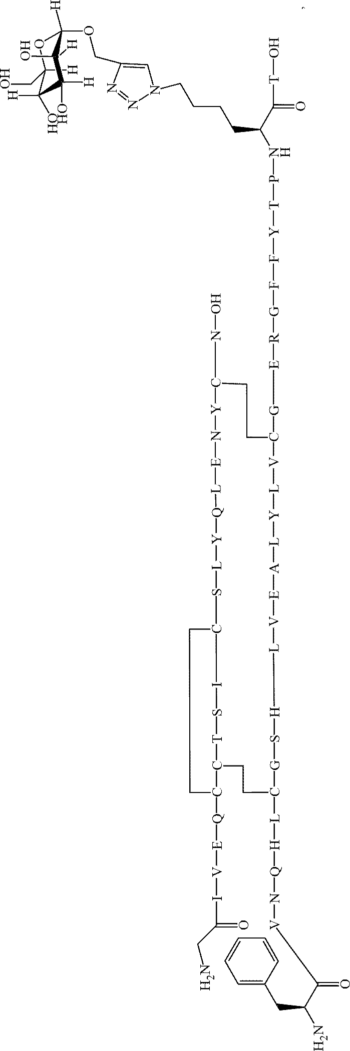 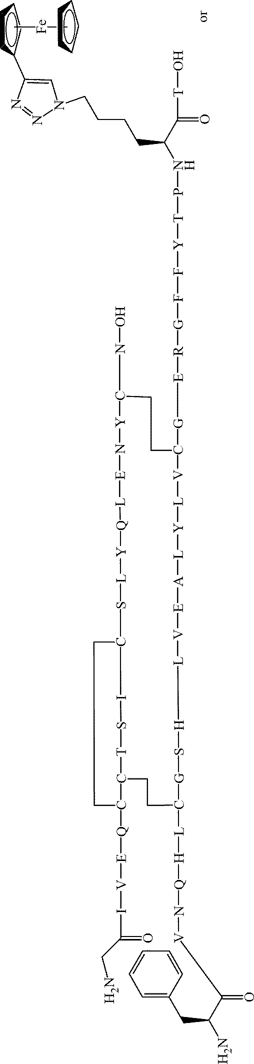 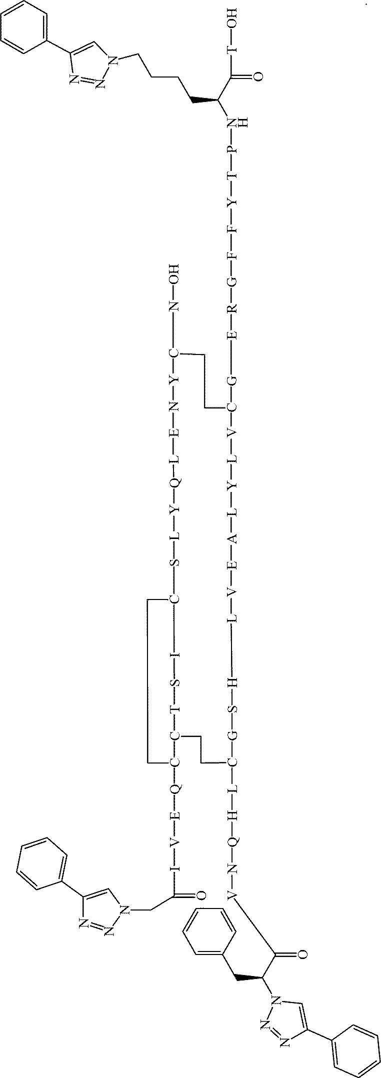
.


SEQUENCE LISTING (9 sequences)

SEQ ID NO: 1 - protein, 21 aa, Homo sapiens
GIVEQCCTSICSLYQLENYCN

SEQ ID NO: 2 - protein, 30 aa, Homo sapiens
FVNQHLCGSHLVEALYLVCGERGFFYTPKT

SEQ ID NO: 3 - protein, 30 aa, synthetic construct
  REGION 1..30: note = Insulin aspart B chain
FVNQHLCGSHLVEALYLVCGERGFFYTDKT

SEQ ID NO: 4 - protein, 30 aa, synthetic construct
  REGION 1..30: note = Insulin lispro B Chain
FVNQHLCGSHLVEALYLVCGERGFFYTKPT

SEQ ID NO: 5 - protein, 21 aa, synthetic construct
  REGION 1..21: note = Insulin glargine A chain
GIVEQCCTSICSLYQLENYCG

SEQ ID NO: 6 - protein, 32 aa, synthetic construct
  REGION 1..32: note = Insulin glargine B chain
FVNQHLCGSHLVEALYLVCGERGFFYTPKTRR

SEQ ID NO: 7 - protein, 21 aa, synthetic construct
  REGION 1..21: note = Insulin A chain mutiens
  VARIANT 8: note = X8 is threonine or histidine
  VARIANT 17: note = X17 is glutamic acid or glutamine
  VARIANT 19: note = X19 is tyrosine, 4-methoxy-phenylalanine, or 4-amino phenylalanine
  VARIANT 21: note = X21 is sparagine or glycine
GIVEQCCXSICSLYQLXNXCX

SEQ ID NO: 8 - protein, 25 aa, synthetic construct
  REGION 1..25: note = Insulin B chain variants
  VARIANT 1: note = X25 is histidine or threonine
  VARIANT 5: note = X29 is alanine, glycine or serine
  VARIANT 6: note = X30 is histidine, aspartic acid, glutamic acid, homocysteic acid, or cysteic acid
  VARIANT 24: note = X31 is proline or lysine
  VARIANT 25: note = X32 is proline or lysine, with the proviso that at least one of X31 or X32 is lysine
XLCGXXLVEALYLVCGERGFFYTXX

SEQ ID NO: 9 - protein, 32 aa, synthetic construct
  REGION 1..32: note = Insulin B chain variants
  VARIANT 1: note = X22 is or phenylalanine and desamino-phenylalanine
  VARIANT 5: note = X25 is histidine or threonine
  VARIANT 9: note = X29 is alanine, glycine, or serine
  VARIANT 10: note = X30 is histidine, aspartic acid, glutamic acid, homocysteic acid, or cysteic acid
  VARIANT 28: note = X31 is aspartic acid, proline, or lysine
  VARIANT 29: note = X32 is lysine or proline
  VARIANT 30: note = X33 is threonine, alanine, or absent
  VARIANT 31: note = X34 is arginine or absent
  VARIANT 32: note = X35 is arginine or absent; With the proviso at least one of X31 or X32 is lysine
XVNQXLCGXXLVEALYLVCGERGFFYTXXXXX